(19) United States
(12) Patent Application Publication
(10) Pub. No.: US 2016/0230180 A1
(43) Pub. Date: Aug. 11, 2016

(54) ENGINEERING THE PLANT ACTIN CYTOSKELETON TO MODULATE CELL WALL PROPERTIES

(71) Applicant: PURDUE RESEARCH FOUNDATION, West Lafayette, IN (US)

(72) Inventor: Daniel B Szymanski, West Lafayette, IN (US)

(73) Assignee: PURDUE RESEARCH FOUNDATION, West Lafayette, IN (US)

(21) Appl. No.: 15/014,504

(22) Filed: Feb. 3, 2016

Related U.S. Application Data
(60) Provisional application No. 62/111,428, filed on Feb. 3, 2015.

(51) Int. Cl. C12N 15/82

(52) U.S. Cl. CPC C12N 15/8225

(57) ABSTRACT

A method for controlling the mechanical properties of the cell wall and polarized growth in a cell that includes selectively manipulating the cell cytoskeleton is presented herein.

### CROSS-REFERENCE TO RELATED APPLICATIONS

The present U.S. patent application is related to and claims the priority benefit of U.S. Provisional Patent Application Ser. No. 62/111,428, filed Feb. 3, 2015, the contents of which is hereby incorporated by reference in its entirety into this disclosure.

### STATEMENT OF GOVERNMENT INTEREST

This invention was made with government support under 1249652 awarded by the National Science Foundation, and MCB 1121893 awarded by the National Science Foundation. The government has certain rights in the invention.

### TECHNICAL FIELD

The present disclosure relates to cell wall heterogeneities and control of plant cell shape.

### BACKGROUND

This section introduces aspects that may help facilitate a better understanding of the disclosure. Accordingly, these statements are to be read in this light and are not to be understood as admissions about what is or is not prior art.

The value of cotton fibers in terms of length and strength is determined, in part, by the rate at which the radius of curvature is refined to a sharp point. High quality fibers refine quickly and elongate as thin long fibers. The cellular and molecular control of this shape refinement are not known, therefore it is not possible to genetically engineer fibers with improved mechanical properties. This is an important technological goal in plants such as cotton because there is little genetic diversity and increased crop value could be most efficiently achieved through genetic engineering. At present, labs are simply searching for factors that affect fiber elongation. For example, gene silencing or inhibitor treatments are conducted and the effects on fiber elongation are assayed. There is no mechanistic understanding of the polarized growth process. There is therefore an unmet need for a method for controlling aspects of the cell such as cell wall thickness, cell wall anisotropy, and cell wall composition.

### SUMMARY

In one aspect, a method for controlling the mechanical properties of the cell wall and polarized growth in a cell is presented, which includes selectively manipulating the cell cytoskeleton. The cell is that of a leaf trichome. The specific gene type controls properties of tip refinement in the cell. The method can further include an engineered protein that modulates the size and position of a microtubule depleted zone, which influences the local properties of the cell wall. The specific gene type includes a genetically engineered protein complex termed the ACTIN-RELATED PROTEIN (ARP) 2/3 that creates actin filaments, the proteins SPIKE1, PIROGI/SRA1, GNARLED/NAP1, BRICK1/HSPC300, and SCAR/WAVE which regulate ARP2/3, ACTIN which polymerizes to form an actin filament network, and microtubule depleted zone in the cell.

### BRIEF DESCRIPTION OF THE FIGURES

FIGS. 1A-1G relate to the geometry of trichome shape change and cell wall strain. FIG. 1A shows the reflected light timelapse imaging of trichome morphogenesis. FIG. 1B shows the branch length, base width, and tip radius of curvature plotted as a function of time. FIG. 1C shows local wall strain analysis using fluorescent beads. Projected image of the tracked bead movement in a 4 hr time course. Yellow dashed lines indicate cell outlines. FIG. 1D shows strain rates as a function of normalized branch length with 0 corresponding to the base and 1 the branch tip. Values are means±SD (n=43), **P<0.01 (Student t-test). FIG. 1E shows microtubule localization visualized with the MBD:GFP reporter. Inset is an enlarged view of the MDZ at the branch apex. Arrows mark branch apex. FIG. 1F shows microtubule angle distributions with 90° defined as transverse to the growth axis. Values are mean±SD (n=14 cells). FIG. 1G shows YFP:CESA6 expressing trichome branch showing linearly aligned CESA6 marked with red arrowheads. Time stamps in FIGS. 1A and 1C indicate hours. Scale bars: 10 μm (A and E), 5 μm (FIGS. 2C and 2G).

FIGS. 2A-2L relate to the finite element model of trichome branch morphogenesis. FIG. 2A illustrates an overview of the FE model: key cell wall parameters (left) and example of trichome branch model growth (right). FIGS. 2B and 2C are plots showing the sensitivity of the branch geometry during growth to fiber dispersion κ and fiber to matrix elastic moduli ratio k₁/E₀. FIG. 2D is a sensitivity map of the width to length slope to k₁/E₀ and κ and its intersection with the observed branch width to length ratio (white plane). The insert shows the κ and k₁/E₀ relationship that led to the experimental aspect ratios. The red point represents experimental κ and the corresponding k₁/E₀. FIG. 2E is a plot showing effects of the TIZ size on the tip refinement. FIG. 2F is a plot showing effects of elastic modulus and thickness gradients on simulated wall strain patterns. nX represents the gradient value with n equal to the base value divided by the tip value. FIGS. 2G and 2H show TEM measurements of wall thickness variation in developing wild-type trichome branches. Blue lines indicate cell wall thickness at branch locations marked by black arrows. FIG. 2I shows wall thickness analyses in a population of wild-type trichome branches using PI fluorescence quantification. Values are mean±SD (n=40). FIGS. 2J and 2K show wall von Mises stresses and maximum in-plane principal logarithmic strains. FIG. 2L is a cell wall synthesis spatial map, in terms of added thickness needed to restore wall thickness after one simulation cycle. Scale bars: 10 μm [left panel in FIGS. 2G and 2I], 100 nm [right panels in FIG. 2G)].

FIGS. 3A-3I relate to the growth and cell wall analysis of an ARP 2/3 null mutant. FIG. 3A shows scanning electron micrographs of wild type and arpc2 trichomes with nascent branches. FIG. 3B shows time-lapse images of soil-grown arpc2 trichomes. Time stamps indicate hours. FIG. 3C is a plot of branch length, base width, and tip radius of curvature as a function of time from FIG. 3B. FIG. 3D shows low and high magnification images of a medial longitudinal section through a developing arpc2 branch. Blue lines indicate cell wall thickness at branch locations marked by black arrows. FIG. 3E shows a regression analysis of wall thickness as a function of distance from the branch tip in FIG. 3D. FIG. 3F shows wall thickness analyses in a population of arpc2 trichome branches using PI fluorescence quantification. Values are mean±SD (n=42). FIGS. 3G and 3H are plots of perimeter length of the MDZ as a function of tip radius of curvature in wild type [FIG. 3G, n=16] and arpc2 [FIG. 3H, n=22] trichomes. FIG. 3I shows a centering of the MDZ relative to branch axis with 1 corresponding to a perfectly centered zone. Values are mean±SD (n=19 for WT, n=26 for arpc2). *P<0.05 (Student ttest). Scale bars: 10 μm [(FIGS. 3A, 3B, and 3F) and left panel in FIG. 3D], 100 nm [right panels in FIG. 3D].

FIGS. 4A-4H relate to the cell wall thickness analysis of developing trichome branches. FIGS. 4A-4C are plots of the TEM analysis of cell wall thickness in wild-type developing trichomes. Sections were obtained from medial longitudinal sections of a young trichome branch and wall thickness was measured directly; the bottom is defined as the trichome branch surface that was closest to the leaf surface and top as the opposite surface; referring to FIG. 4A, the wall thickness profile is of the opposite wall from the cell shown in FIG. 2G. FIGS. 4B and 4C relate to wall thickness profiles of both wall segments taken from a TEM analysis of a second independent trichome. Cell wall thickness gradients are consistently present in wild-type trichome branches. FIGS. 4D-4G relate to the control experiment to indicate that the PI signal gradient in FIG. 2I is not an optical artifact of the cell geometry. The plasma membrane was stained with FM1-43 in developing wild type (FIG. 4D) and arpc2 (FIG. 3F) trichomes. Scale bars, 10 μm. Regression analysis of mean values shows a negative correlation between signal intensity and location along the branch in both wild type [(FIG. 4E); n=14) and arpc2 [(FIG. 4G); n=22]. Values are mean±SD. FIG. 4H shows plots of FE modeling simulations of the effects of cell wall thickness gradients on tip-biased anisotropic growth. Wall thickness gradient values ranged from 50 to 250 nm and were varied every 2 μm along the branch length.

FIGS. 5A-5G relate to the local strain rates, cell wall thickness and texture of the null ARP2/3 mutant arpc2. FIG. 5A shows strain rates of an arpc2 branch as a function of normalized branch length. Premature papillae on the elongating branch shown in FIG. 3B were used as fiducial marks to measure local axial strain. Values are means±SD (n=14). FIGS. 5A-5D show TEM analysis of cell wall thickness in arpc2 developing trichomes. Sections were obtained from medial longitudinal sections of a young trichome branch and wall thickness was measured directly. The wall thickness profile of the opposite wall from the cell shown in FIG. 5D (B). Wall thickness profiles of top and bottom wall segments taken from a TEM analysis of a second independent trichome (FIGS. 5C and 5D). Cell wall thickness gradients are not present and there is an increase in the local variability of cell wall thickness. As shown in FIG. 5E, cellulose microfibrils have a transverse arrangement in young arpc2 trichomes. Glancing section of the cell wall reveals highly aligned microfibrils. Inset panels are magnified views of FIG. 5E showing fiber texture in glancing (above) and more transverse (below) sections. Scale bars, 1 μm (left panel), 0.5 μm (right panels). FIG. 5F shows a transverse microtubule localization of the arpc2 mutant branch visualized with the MBD:GFP reporter. An arrow marks the MDZ. Scale bar, 5 μm. FIG. 5G shows microtubule angle distributions, with 90° defined as transverse to the growth axis. Values are mean±SD (n=14 cells).

FIGS. 6A-6I relate to a demonstration of the functionality of ARPCS:GFP, analysis of BRK1:YFP localization, and an analysis of the altered organelle motility patterns in arpc2. FIGS. 6A-6F show a GFP-fusion to the C-terminus of ARPCS is fully functional. ARP2/3:GFP rescued the arpc5 mutant trichome phenotype. Mature leaf trichomes on wild type (FIGS. 6A and 6B), and arpc5 (FIGS. 6C and 6D) showing distorted phenotype. ARP2/3:GFP expressed in arpc5 background rescued the trichome phenotype (FIGS. 6E and 6F). Scale bars, 1 mm in (FIGS. 6A, 6C and 6E), 100 μm in (FIGS. 6B, 6D and 6F). FIGS. 6G and 6H show ARPCS:GFP is present as an intact 44 kDa fusion protein in cells and does not affect the membrane association or level of another ARP2/3 subunit ARP3. Proteins from wild type and two independent ARPCS:GFP transgenic lines (CSGFPt2-8 and t2-4) were probed with anti-GFP (FIG. 6G). Endogenous ARP3 is not overexpressed in the ARPCS:GFP lines. PEPC is used as loading control (FIG. 6H). FIG. 6I shows BRK1:YFP accumulates at the branch apex, marked with an arrow.

FIGS. 7A-7O show ARP2/3-generates apical actin meshwroks within the microtubule-depletion zone. FIGS. 7A-7C show ARP2/3 localization in the rescued line ARPC5:GFP;arpc5 (FIG. 7A); ARP2/3 localization in arpc2 (FIG. 7B); ARP2/3 localization in the W/SRC mutant nap1 (FIG. 7C). FIGS. 7D and 7E show colocalization of ARP2/3 and actin meshworks at the branch apex. ARP2/3, phalloidin-labeled actin filaments, and merged images (FIG. 7D). Intensity plot of ARP2/3 and actin signals along the arrow shown in the inset of (FIG. 7E). FIGS. 7F-7H show apical actin meshworks labeled by phalloidin in wild type (FIG. 7F), and their absence in arpc5 (FIG. 7G) and arpc2 (FIG. 7H). Arrows label actin bundles; arrowheads, branch apex. FIG. 7I shows particle tracking of Golgi on actin bundles labeled with LifeAct:mCherry. Tracked Golgi are labeled with a colorized path; arrows indicate the direction of the motility. FIGS. 7J and 7K show projections of Golgi motility over a 2 min interval in wild type (FIG. 7J) and arpc2 (FIG. 7K). A circle in (FIG. 7K) highlights Golgi with minimal movement. FIG. 7L shows directionality of Golgi movement is reduced in arpc2. Values are mean±SE (n>150), **P<0.01 (Wilcoxon test). FIG. 7M shows ARP2/3 functions within the MDZ. ARP2/3, microtubules (mCherry:TUA5) and merged images. Lines indicate perimeter lengths of ARP2/3 signal and the MDZ. FIG. 7N shows geometry of the MDZ, ARP2/3 domain, and the optimal TIZ as a function of tip radius. The optimal TIZ was computed from (FIG. 7O) using adaptive iteration. FIG. 7O shows FE simulations using variable TIZ geometries derived from experimental data or a simulated optimal TIZ that generated the best fit to the observed tip refinement pattern. Scale bars, 5 μm.

FIGS. 8A and 8B show localization of microtubules (FIG. 8A) and actin (FIG. 8B) in cotton fibers that were fixed and labeled at 1 DPA, MDZ, microtubule-depletion zone. Dark arrowheads point to longitudinal actin; white arrowheads point to an apical region with increased actin signal that contacts the bundles. Scale bars=5 μm.

FIGS. 9A and 9B show time-lapse imaging of 2 DPA cotton fibers in the process of tip-refinement. FIG. 9A is a merged image of the fluorescent beads and a bright field image at the initial time point. FIG. 9B is an image of the fiber cell 12 hours later, with the tracked particle paths overlaid on the image. Centroids of circles mark the particle positions at specific time points. Scale bars=10 μm.

FIG. 10 shows the labeling of Arabidopsis cell cultures with ¹³Cxylose. Metabolites were analyzed following the pulse labeling and the relative content of 13C determined, and illustrates that xylose is predominantly metabolized by direct conversion to UDP-xylose through the action of sugar kinases and pyrophosphorylases, and only a minor flux goes through the pentose phosphate pathway.

FIG. 11A shows abundance profiles of 16 subunits of the proteasome separated by SEC (left) and IEX (right).

FIG. 11B shows the result of a clustering analysis of 1593 cytosolic proteins into 150 different clusters. This single cluster contains 15 proteasome proteins ([X]) and four other unknown proteins (♦).

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In response to the unmet need, herein disclosed is a method demonstrating how an engineered cytoskeletal protein can be created to control properties of the actin and microtubule cytoskeleton in plants to control cell wall thickness, cell wall anisotropy, and cell wall composition.

We disclose new mechanisms by which an apical actin meshwork influences two cell wall parameters that dictate the patterns of polarized diffuse growth. The ARP2/3-generated meshwork influences wall thickness gradients at the spatial scale of the cell by positioning a network of core actin bundles that organize a bidirectional trafficking system for distributed secretion. The thickness gradient introduces an apical bias to cell elongation and promotes rapid cell elongation. The identity of the actin-dependent secreted cargo remains to be determined. ARP2/3 is activated within an apical microtubule-depleted zone to modulate its size and position. Our results indicate that the local geometries of the ARP2/3 activation domain, the MDZ, and an isotropic cell wall domain mediate smooth tip refinement. It is possible that intracellular signaling and the polarization of cytoskeletal and membrane domains are sufficient to control local wall properties during tip refinement. Alternatively, mechanical properties of the cell wall may feed back on intracellular pathways to coordinate events in the cytoplasm with the status of the extracellular matrix. In plants, the SPK1 and the W/SRC complex transmit activating ROP GTPase signals to ARP2/3; a further analysis of this conserved control module will provide important knowledge about the cellular mechanisms of localized ARP2/3 activation and cell shape control.

The herein disclosed method is superior to the prior art in the following ways. First, it identifies plausible spatial and material properties of the cell wall that can lead to efficient tip refinement in a model system that is directly applicable to cotton fiber elongation. Second, it creates a computational model using Abacus software that can be used to simulate growth and drive experimental design. Third, we have in hand 14 known genes that control the tip refinement process. We know their localization and function in terms of localized actin polymerization. Leaf trichomes protect the growing leaf against insect attack, and like the important textile crop of single-celled cotton fibers, use the microtubule and actin cytoskeletons to control highly polarized shape change. These specialized cells, like all others that use a diffuse growth mechanism, modulate cell wall properties to convert an isotropic turgor force into an irreversible growth response. Microtubule-dependent cellulose microfibril patterning is one central feature of polarized diffuse growth; however, the means by which the actin cytoskeleton influences cell shape change is not known. The distorted epidermal morphology mutants identified an evolutionarily conserved actin filament nucleation pathway, in which the WAVE/SCAR regulatory complex (W/SRC) positively regulates the actin filament nucleation actin-related protein (ARP) 2/3. Herein we integrate live cell imaging and computational analyses of the cell wall to determine how an ARP2/3-generated apical actin meshwork influences cell wall properties that define the geometry of shape change.

Creation of a Plausible Finite Element Model:

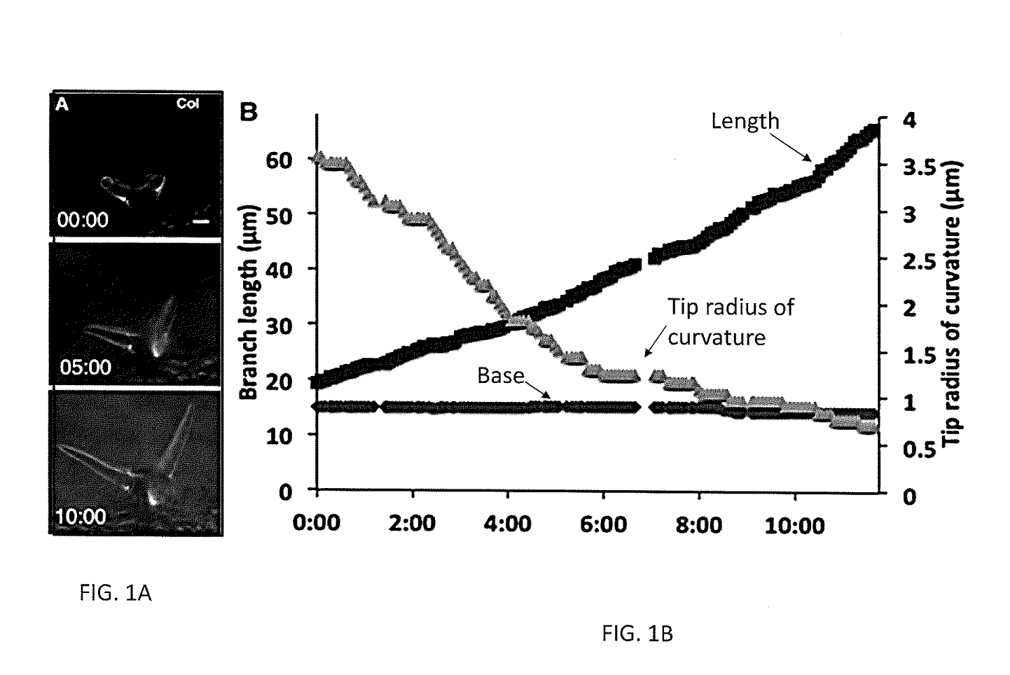

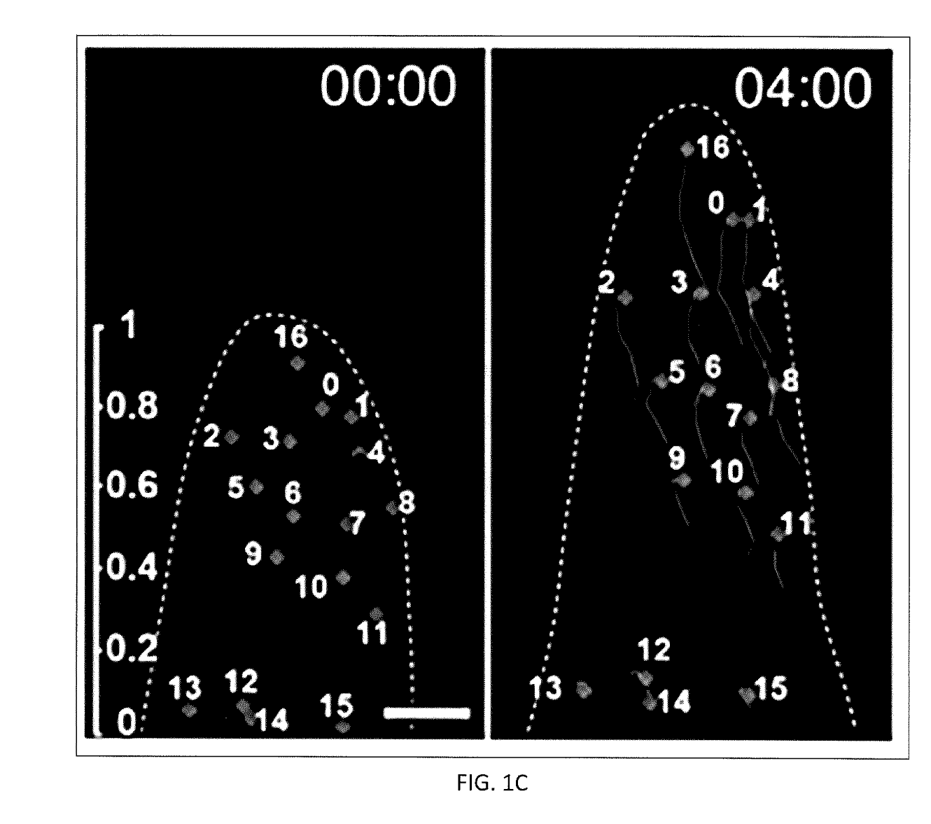

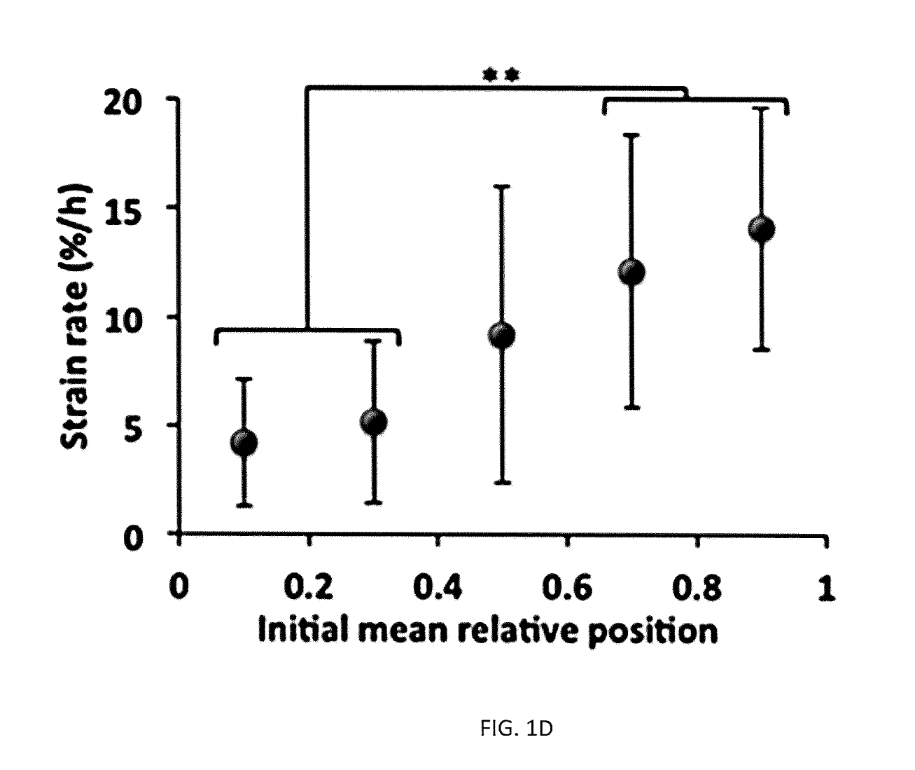

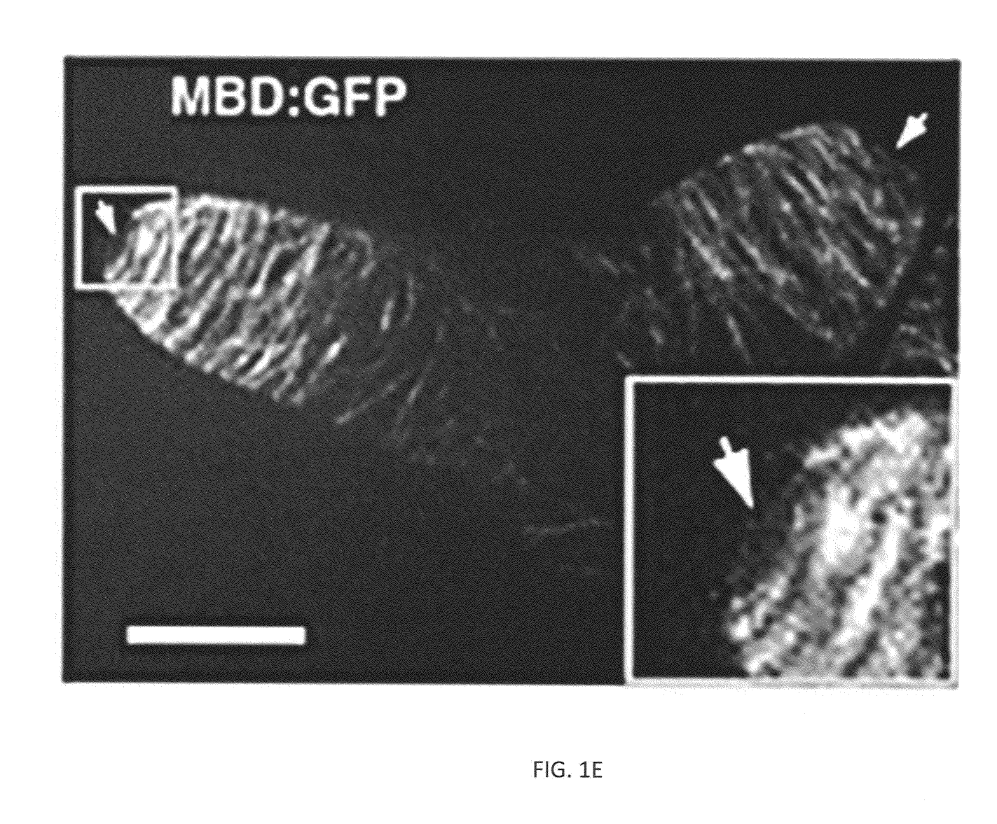

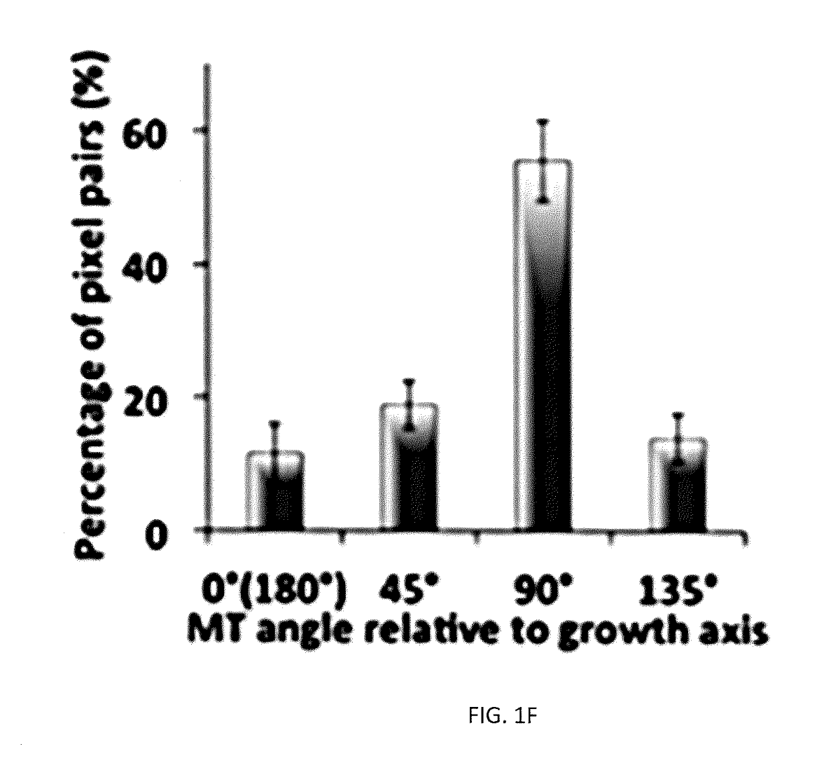

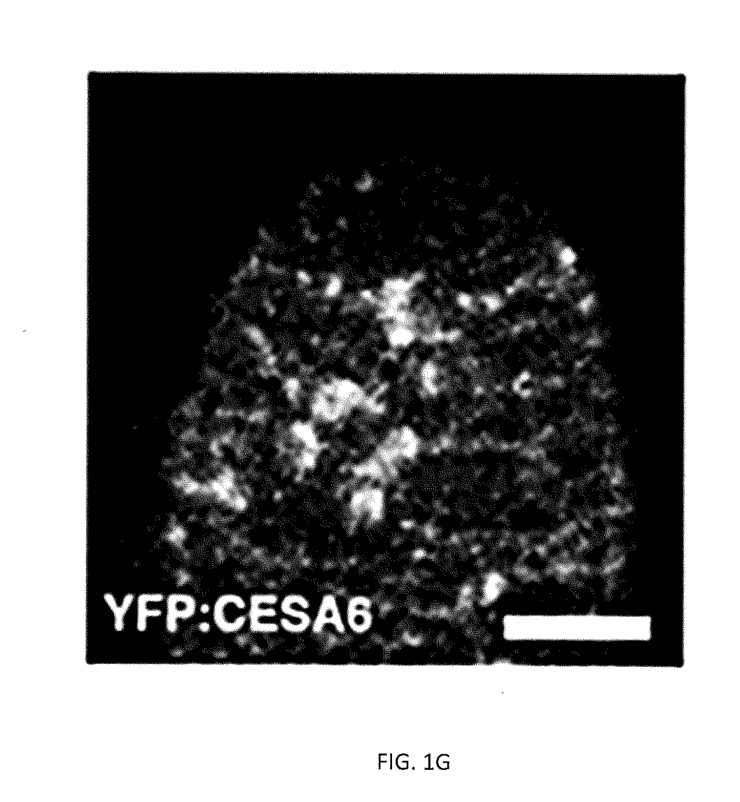

Branch elongation and tip refinement are defining features of trichome morphogenesis. In order to parameterize a finite element (FE) model of the cell wall and branch elongation, we conducted a series of reflected light time-lapse imaging experiments. Branch diameter at the base was nearly constant during growth, as would be expected for tip growth. However, the radius of curvature at the tip decreased from ˜4 μm to ˜0.5 μm as the branch elongated (FIGS. 1A and 1B), and the branch elongation rates increased from 2.3±0.8 μm/hr at early stages to 7.9±1.1 μm/hr in branches at later stages. These observations were consistent with previous evidence for a diffuse growth component to trichome morphogenesis. To measure wall strain patterns directly, fluorescent beads were used as fiducial marks along the branch axis (FIG. 1C). Particle tracking of bead pairs detected an axial strain gradient, in which distal regions of the branch grew ˜3 times faster than the branch base (FIG. 1D). Therefore, the early stages of branch elongation include highly polarized anisotropic diffuse growth and tip refinement to a final radius of 0.61±0.12 μm (n=20). Our analysis captured the significant shape change during branch development because mature branches that exceed 200 μm in length have similar length to width aspect ratios and tip geometry.

Organized microtubules and cellulose microfibrils are required for polarized branch growth. To quantify the degree of potential microtubule-based microfibril ordering, we measured the microtubule angle distribution relative to longitudinal axis of the cell (FIG. 1F). As expected, both microtubules and CESA had a similar transverse alignment (FIGS. 1E and 1G). However, the branch apex always contained an obvious microtubule-depleted zone (MDZ) that lacked patterned CESA localization, indicating that the branch apex wall had a more isotropic character compared to the flank.

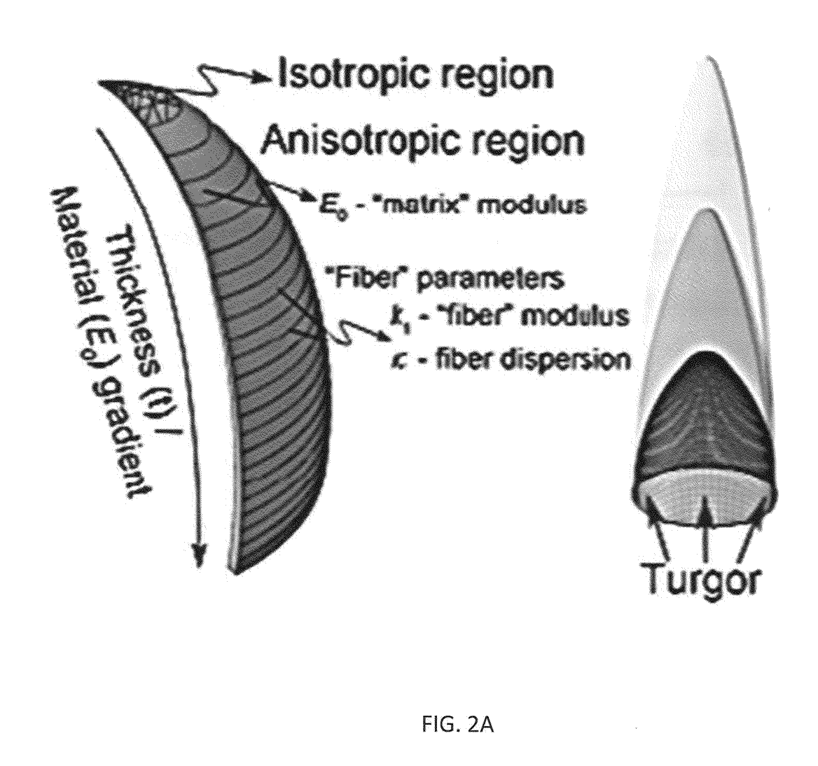

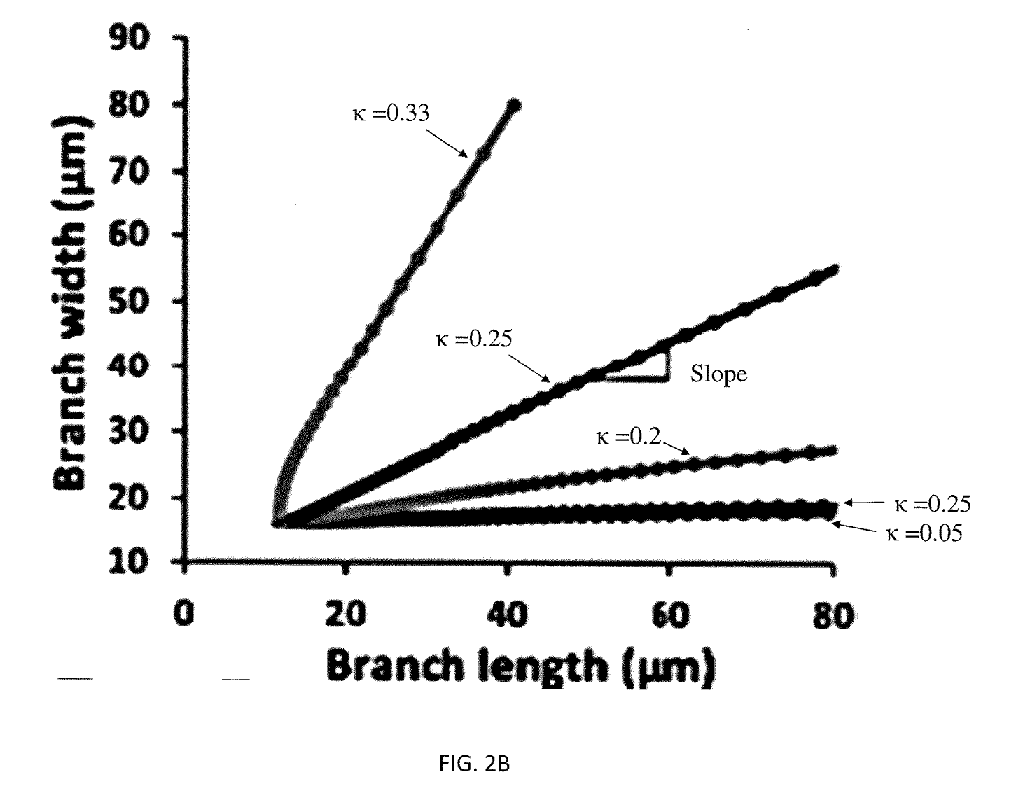

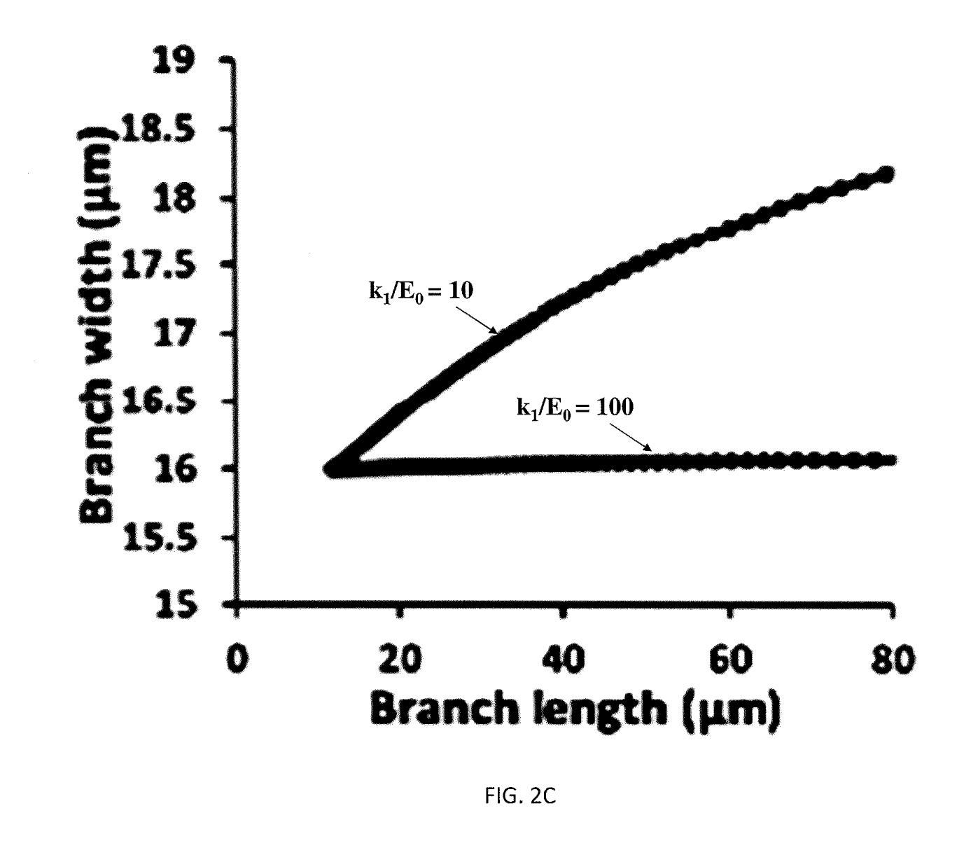

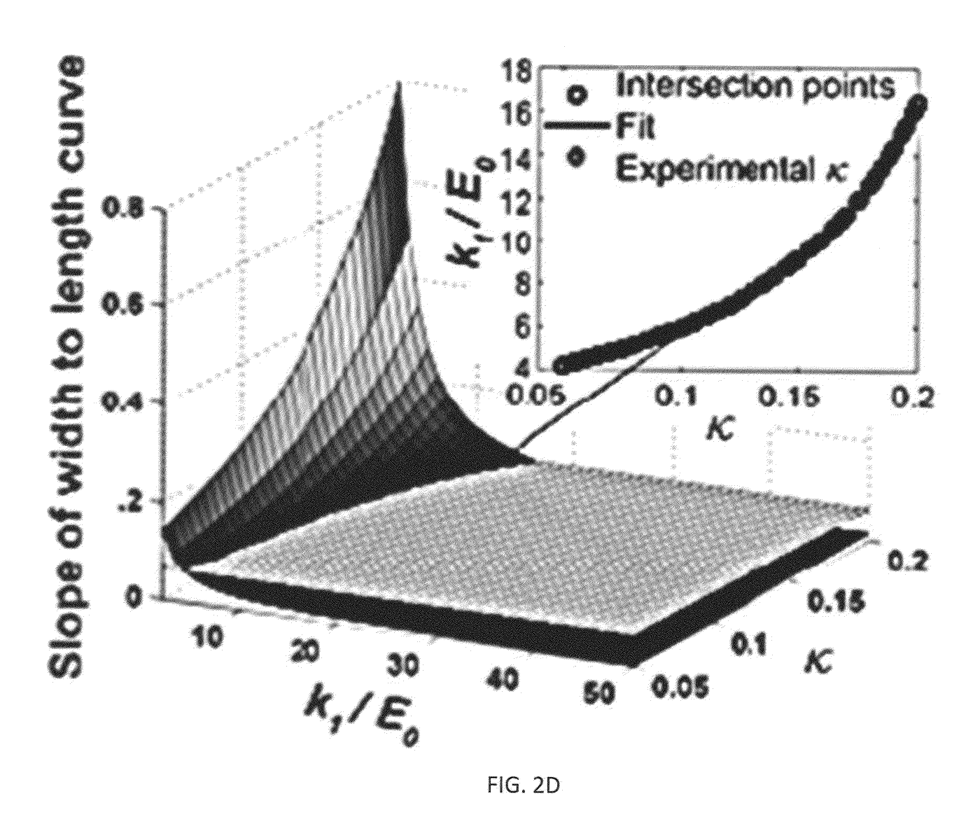

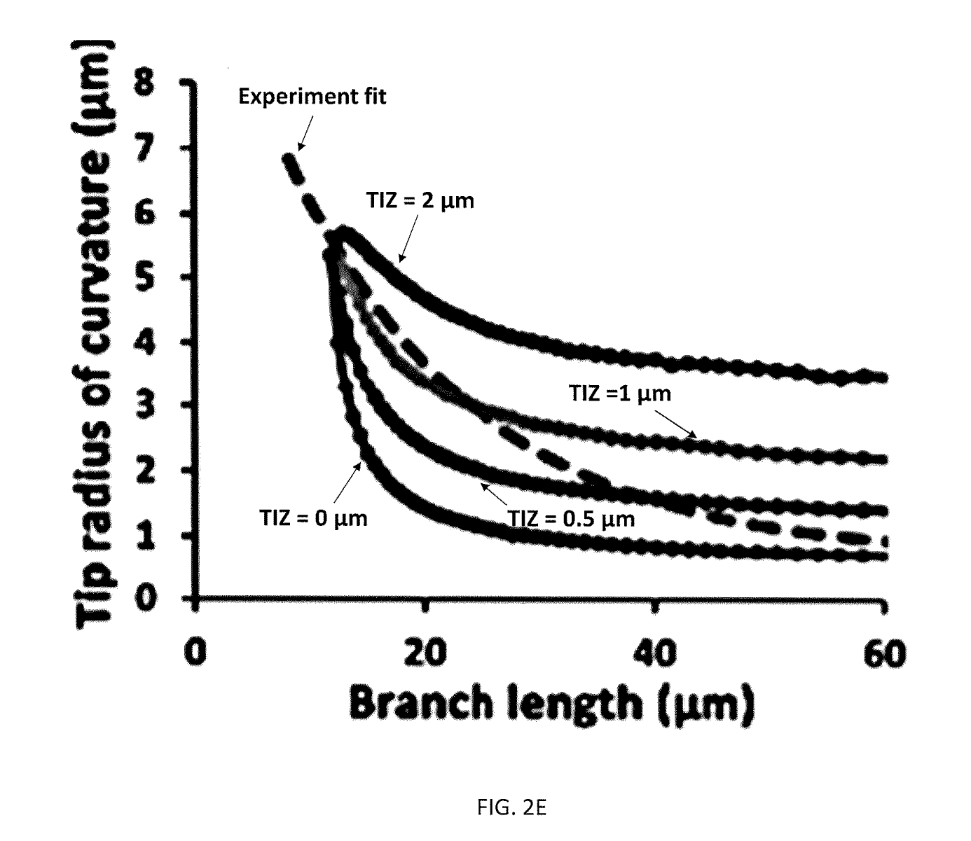

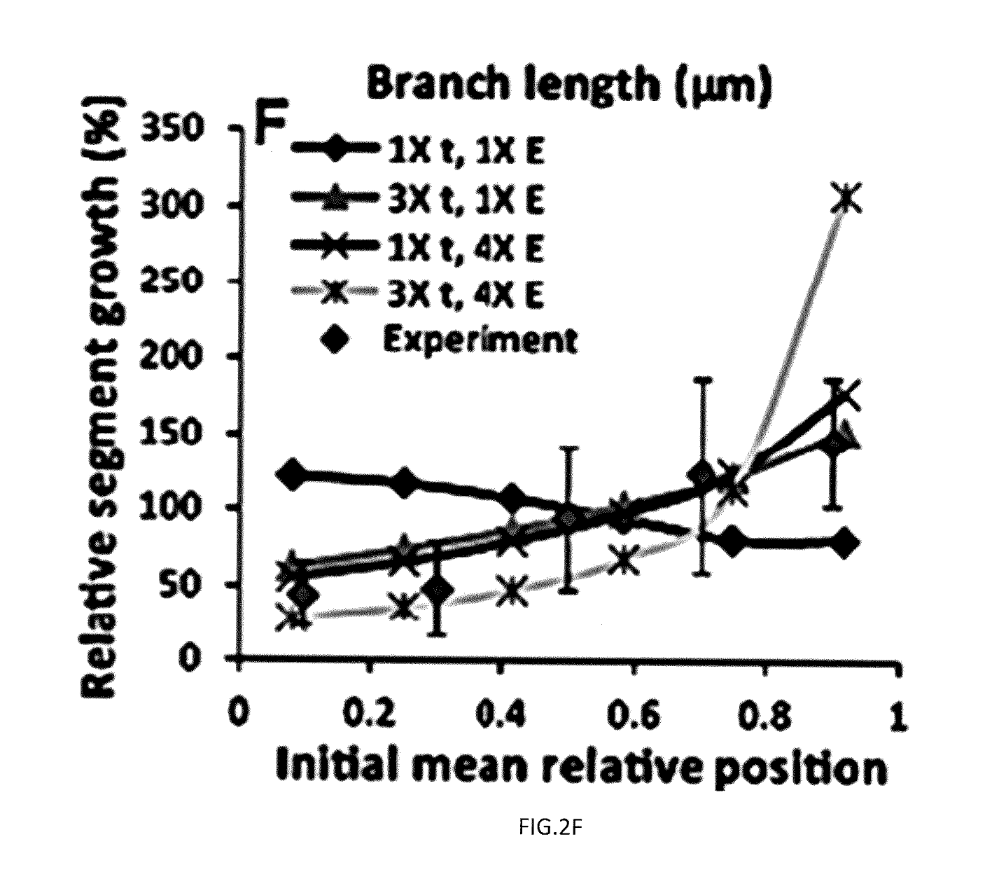

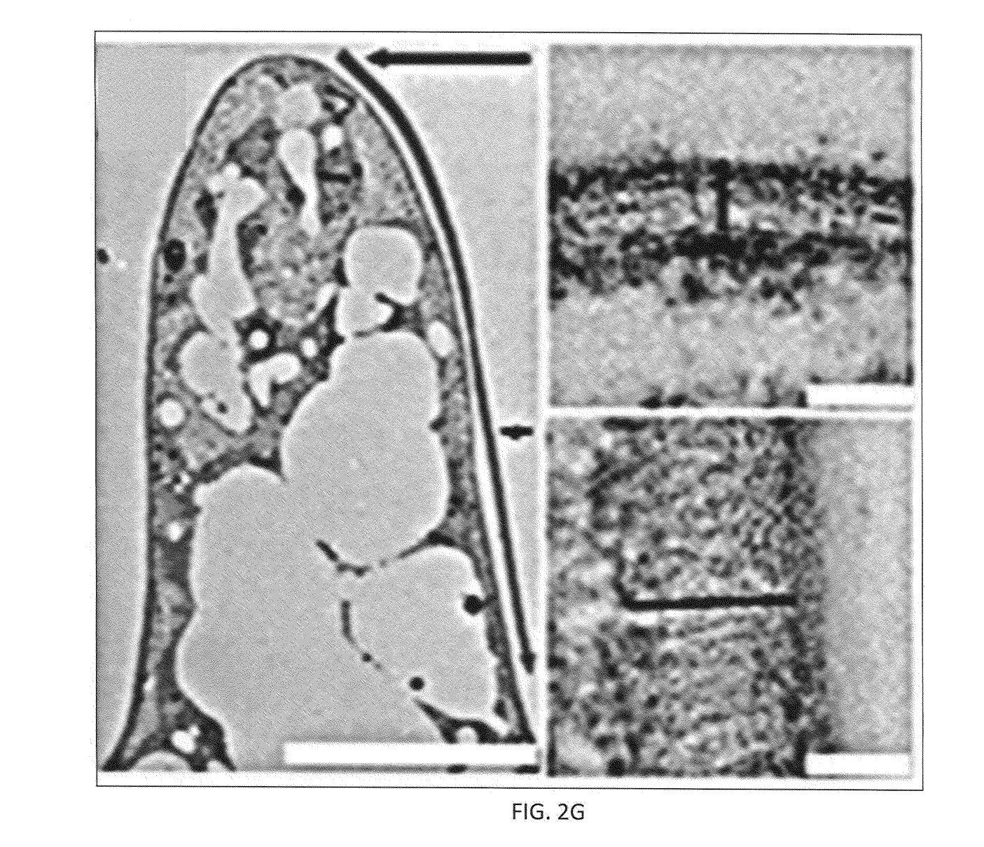

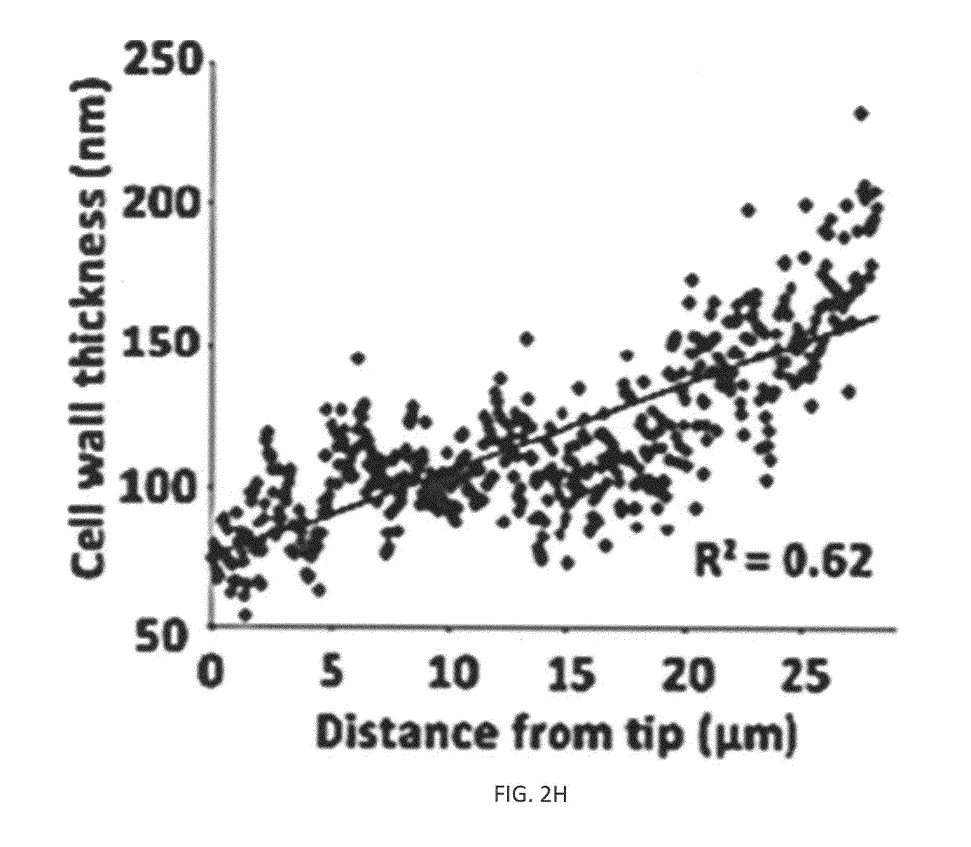

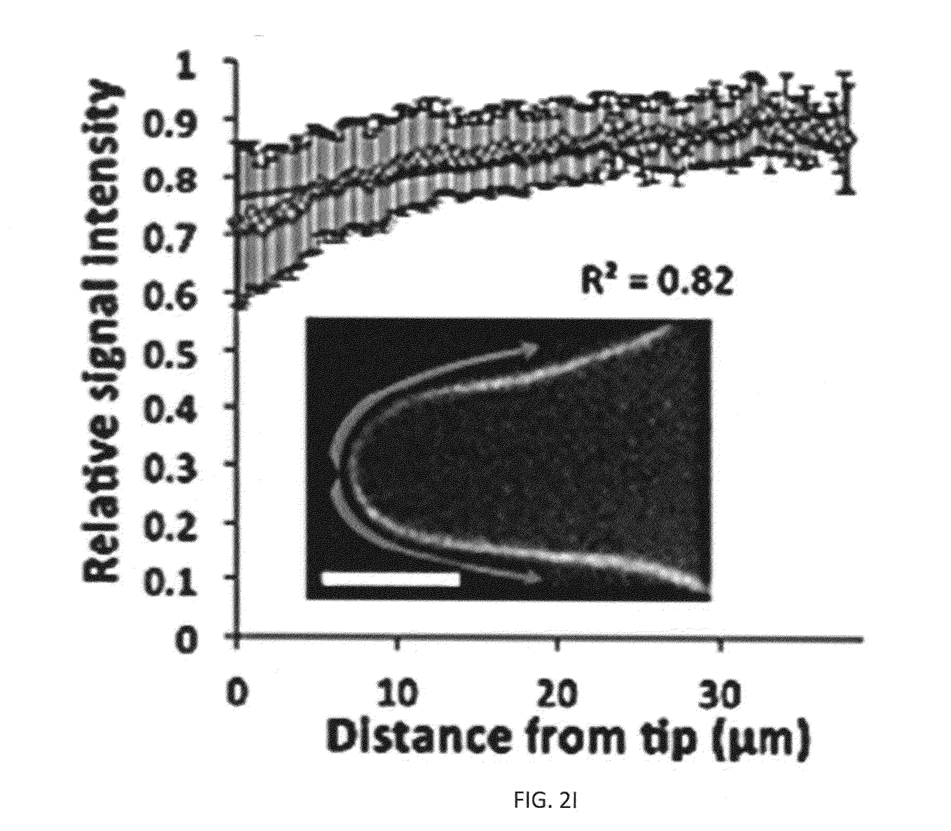

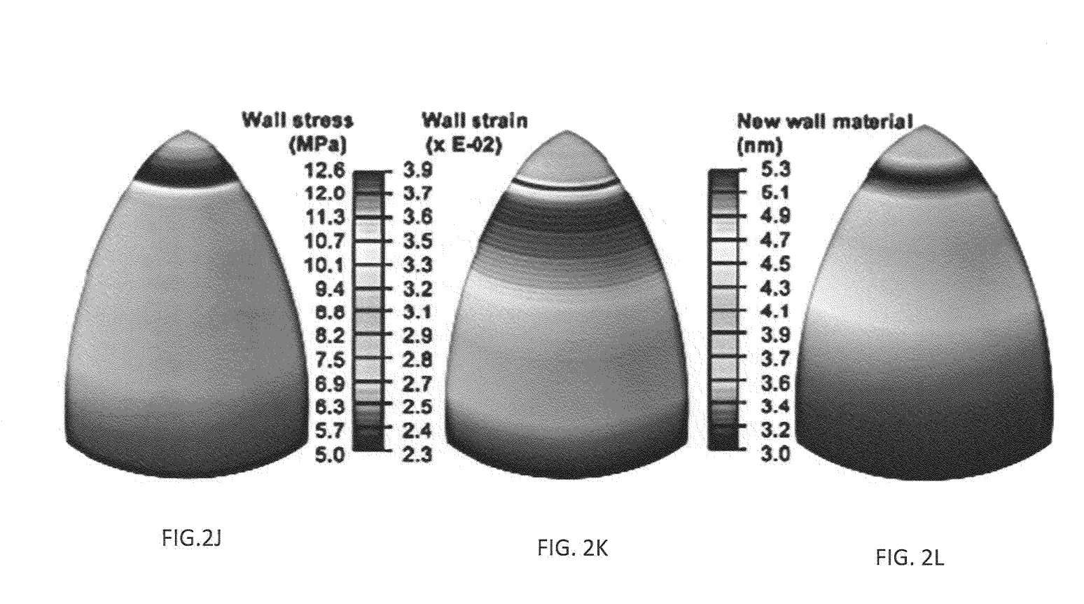

FE modeling has been used to analyze the shape change of thin-walled pressurized plant cells, and can incorporate realistic material models of fibers reinforcing a matrix. Spatial heterogeneities in the model were introduced by subdividing the shell longitudinally into multiple sections with varying properties (FIG. 2A). FE simulations were able to identify wall parameters that affected the rate of cell expansion and growth anisotropy (Table 1). For example the degree of fiber orientation strongly affected anisotropic expansion (FIG. 2B). The measured microtubule angle distributions (FIG. 1F) provided experimental support for highly ordered fibers with κ=0.141±0.029, which could generate branches with the observed high length to width aspect ratios (FIG. 2B). Anisotropic branch expansion was also sensitive to the ratio of fiber to matrix elastic moduli k₁/E₀ of the wall (FIG. 2C). A sensitivity analysis of the branch growth geometry to combinations of k₁/E₀ and κ parameters revealed the potential solution space (FIG. 2D). The experimentally observed values of branch width to length slope of 0.064±0.026 and κ provided the value of k₁/E₀=8.4.

TABLE 1
The effect of model parameters on trichome branch growth patterns. The arrows
indicated an increase in the chosen parameter (↑) and an increase (↑)
or decrease (↓) in resulting width or radius of a 50 μm branch. The size
of the tip isotropic zone (TIZ) has a substantial effect. The fiber dispersion
parameter and thickness gradient are the two additional parameters of importance.

| Model parameter | | Effect on width | | Effect on tip radius | |
|---|---|---|---|---|---|
| (range of values considered) | Physical meaning | (when L = 50 μm) | | (when L = 50 μm) | |
| ↑ k₂ (0.01-100) | Material parameter | <1% | | <1% | |
| | related to stress- | | | | |
| | strain stiffening | | | | |
| ↑ κ (0.05-0.25) | Fiber dispersion | ↑ 121% | | ↑ 11.5%* | |
| | parameter | | | | |
| ↑ E₀ (40-400 MPa) | Elastic modulus of | <1%, | g.p.c | <1% | |
| | isotropic part of | | | | |
| | the cell wall | | | | |
| ↑ k₁/E₀ (10-100) | Fiber-to-matrix | ↑ 8.5% | | ↓ <3%* | |
| | moduli ratio | | | | |
| ↑ TIZ (0-2 μm) | Tip isotropic | <1% | | ↑ 374% | |
| ↑ P (0.2-1 MPa) | Turgor pressure | <2%, | g.p.c. | <2% | |
| ↑ t (0.1-0.5 μm) | Cell thickness | <1%, | g.p.c. | ↑ 50% | (aniso tip) |

| | | | | |
|---|---|---|---|---|
| | | | ↓ 9% | (iso tip) |
| ↑ E₀ gradient (1X-4X) | Spatial gradient | ↓ <4% | ↓ 36.7% | (aniso tip) |
| | of parameter (cell | | ↓ 22.5% | (iso tip) |
| ↑ t gradient (1X-5X) | thickness and/or | ↓ <4% | ↓ 50.5% | (aniso tip) |
| | E₀) from tip to | | ↓ 26.5% | (iso tip) |
| ↑ combined E₀ and t | base | ↓ <2.5% | ↓ 7.5% | (iso tip) |
| gradient (5X t, 1X-4X E₀) | | | | |

*Tip radius increases when the wall material is close to isotropic (κ > 0.25, k₁/E → 1)
g.p.c.—growth per cycle

Importance of a Tip Isotropic Zone and a Cell Wall Thickness Gradient:

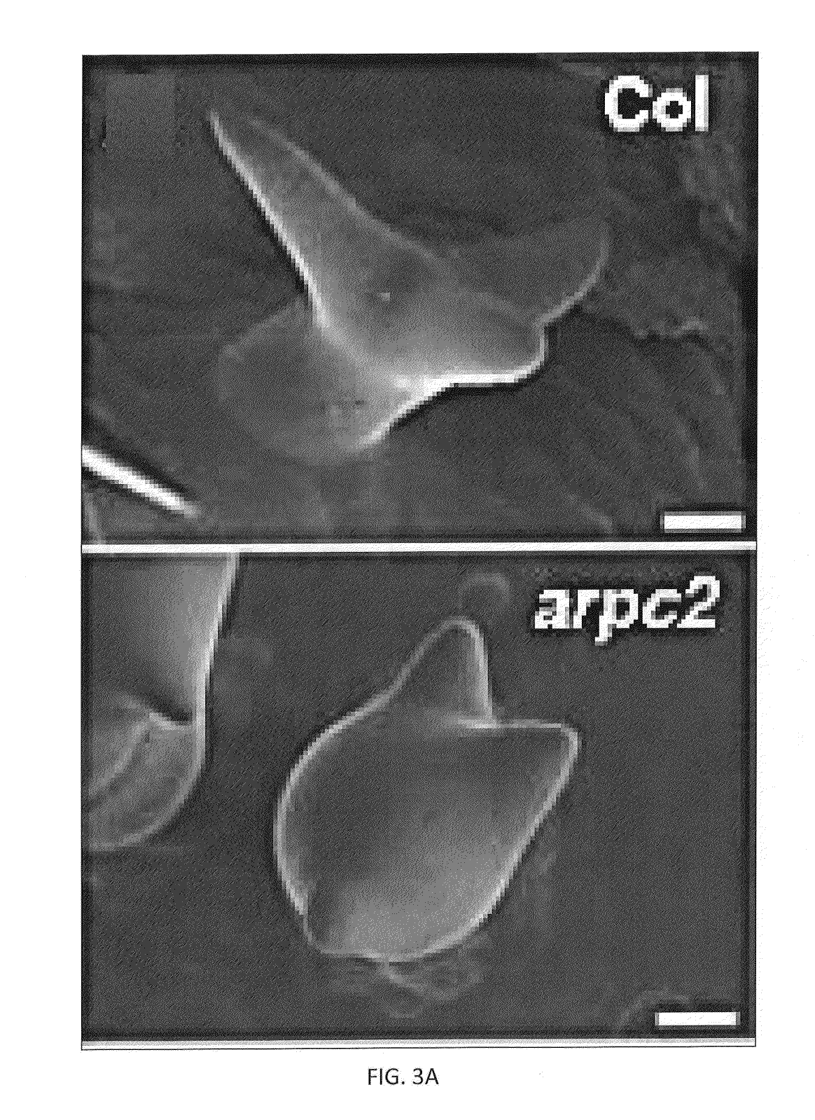

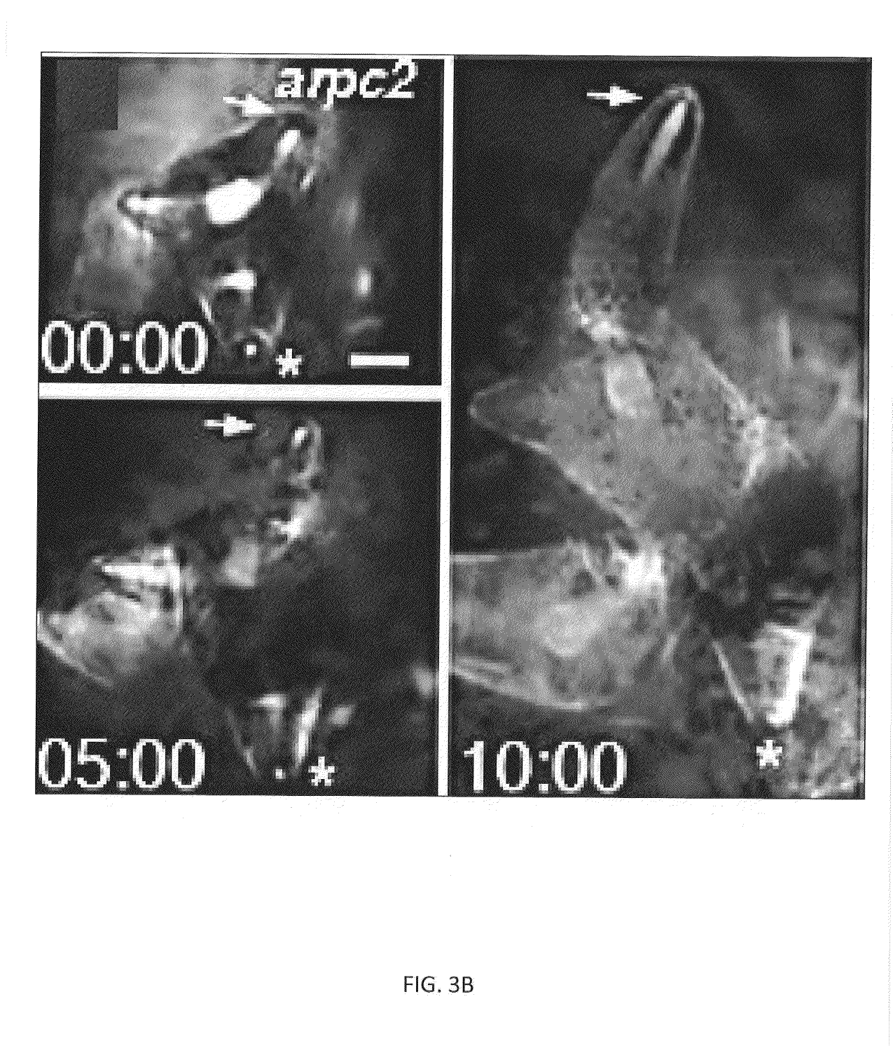

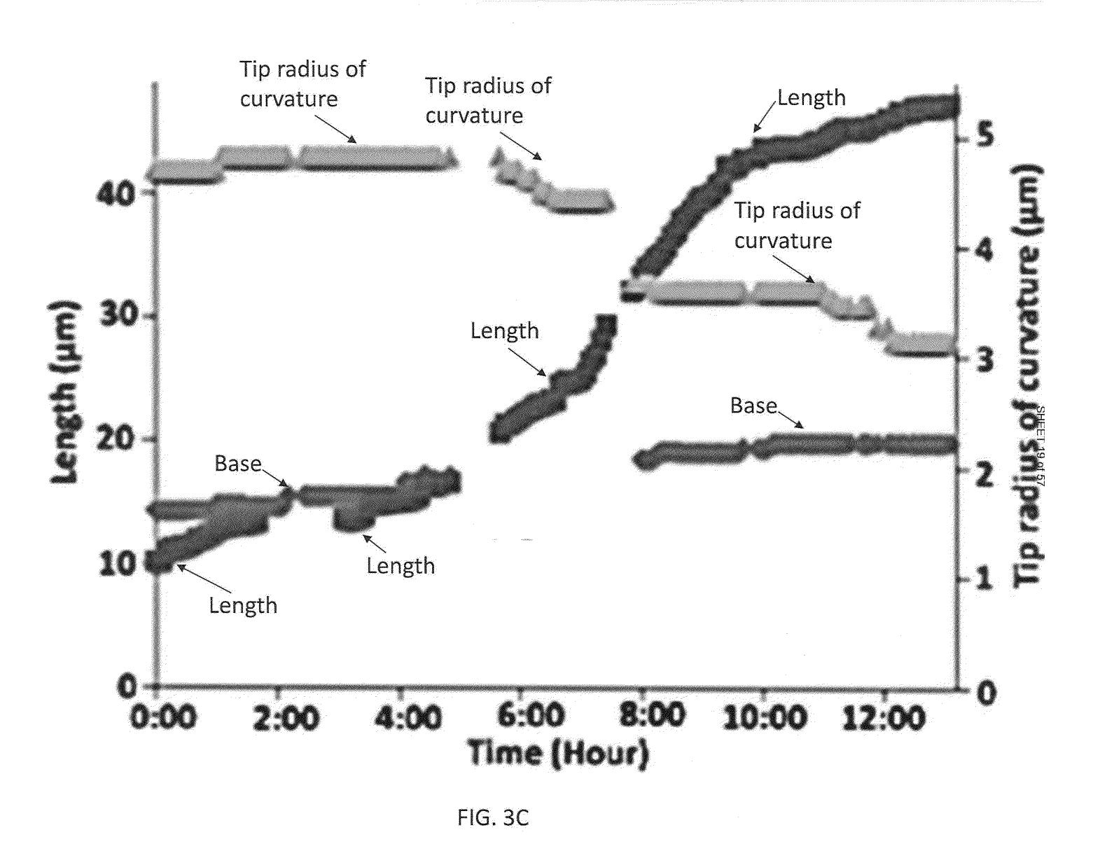

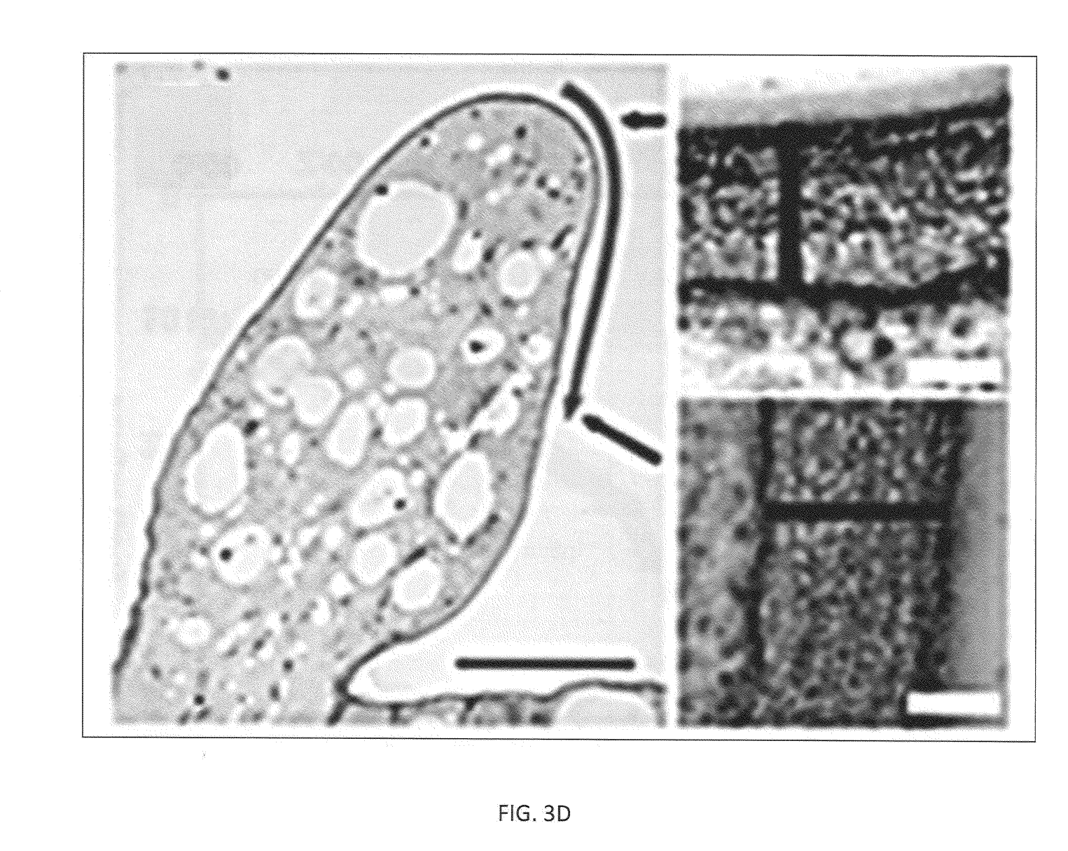

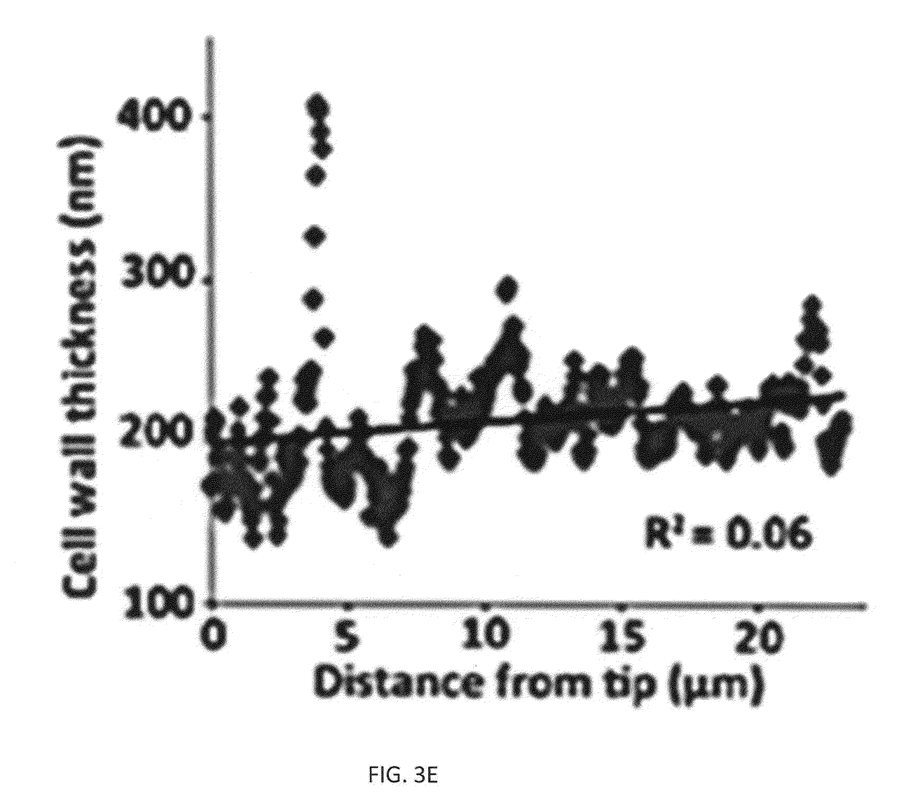

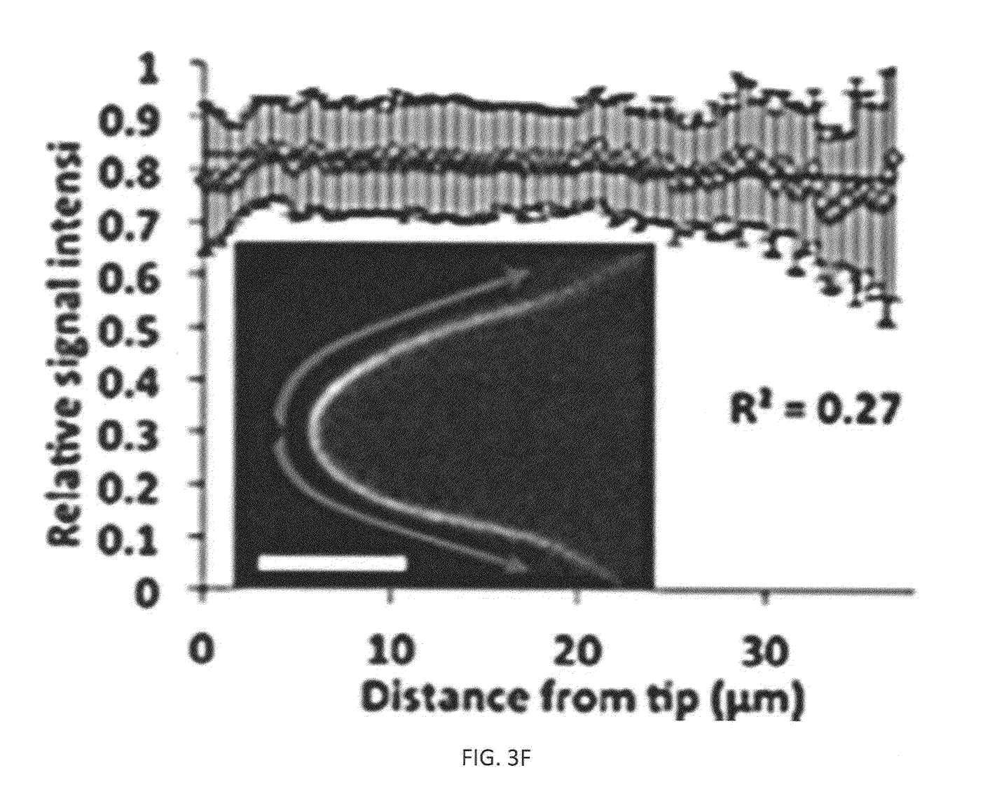

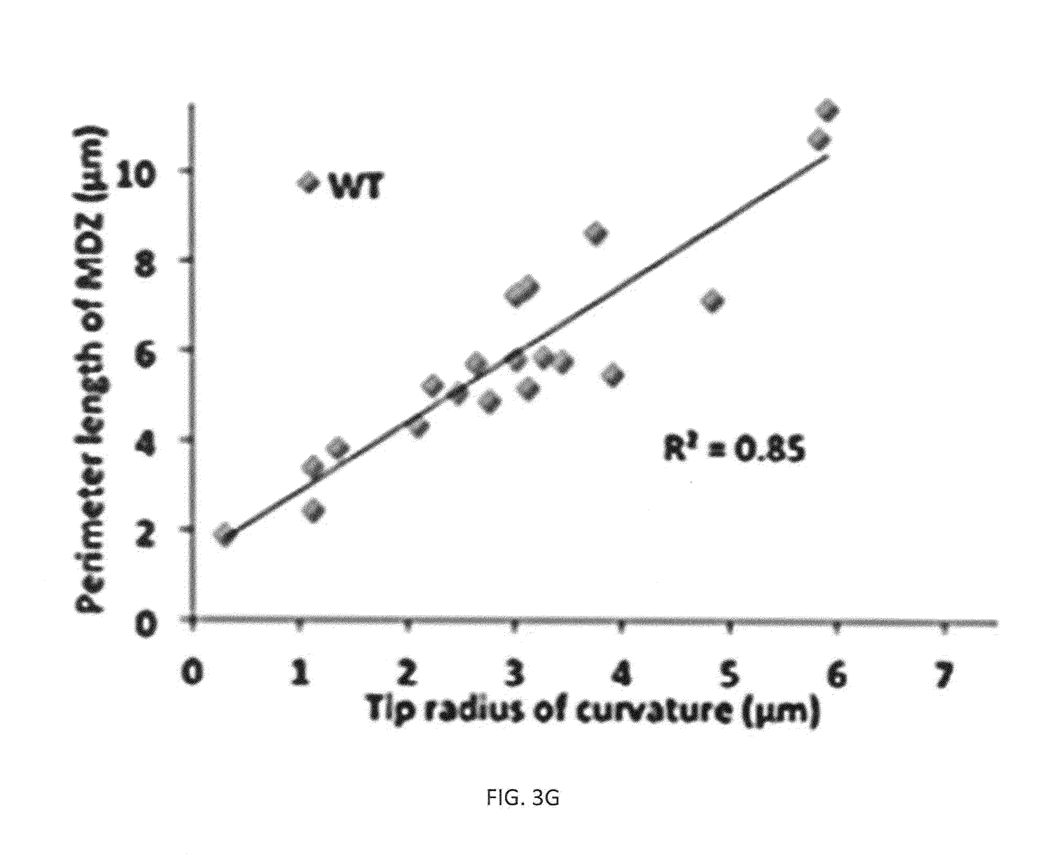

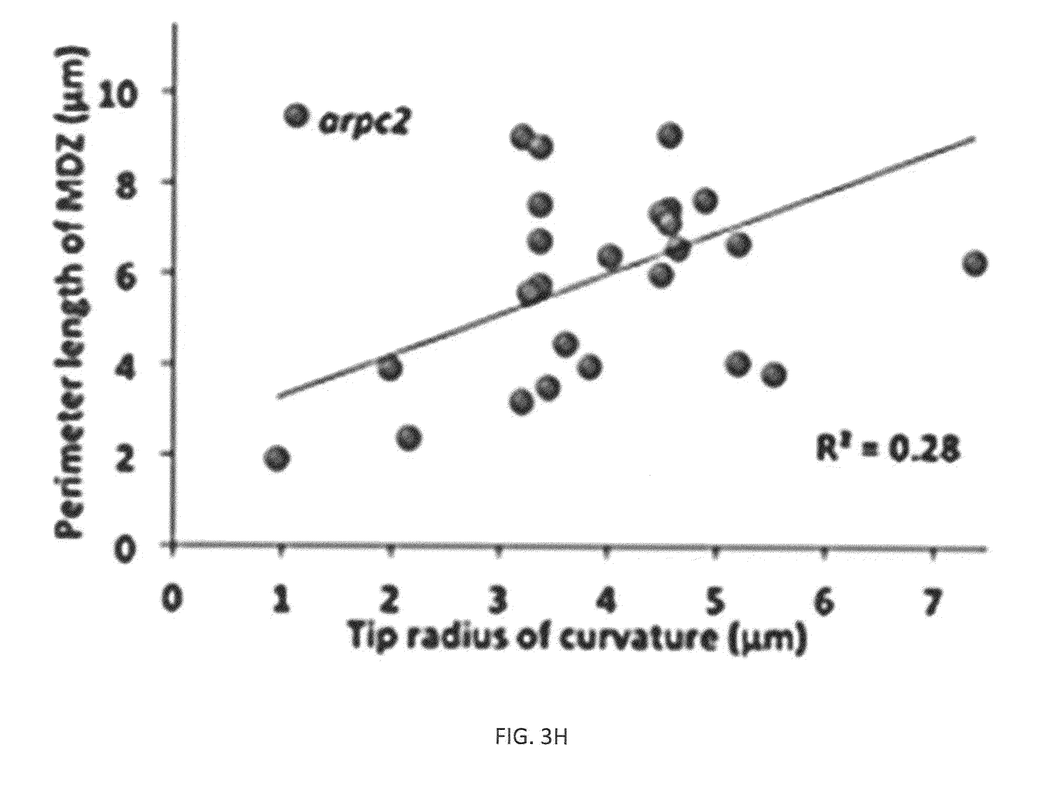

These combinations of k₁/E₀ and κ could reproduce the observed global aspect ratios of branch growth, but could not generate the reproducible patterns of tip refinement (FIG. 2E) and unequal axial strain (FIG. 2F) that were measured in the time-lapse analyses. Live cell measurements indicated that reductions in tip radius and MDZ size were highly correlated, and that the MDZ was efficiently centered on the branch axis (FIGS. 3G and 3I), suggesting that an isotropic branch tip was involved in cell shape control. The FE simulations showed that the trajectories of simulated tip refinement were very sensitive to the size of the tip isotropic zone (TIZ). Intermediate TIZs yielded better fits to the observed growth patterns; however, no single value performed the best for all simulation cycles (FIG. 2E). The modeling results and the coupled geometry of the MDZ and the branch tip radius of curvature suggest that the smooth progression of tip refinement includes modulation of the size of an apical cell wall domain with isotropic mechanical properties (see below). Finally, the observed apical to basal strain gradients that were detected in the bead labeling experiments (FIGS. 1C and 1D) were studied. FE simulations that included fiducial marks could not reproduce the experimentally measured strain gradients unless a proximo-distal gradient in either elastic modulus or wall thickness gradient was introduced (FIG. 2F).

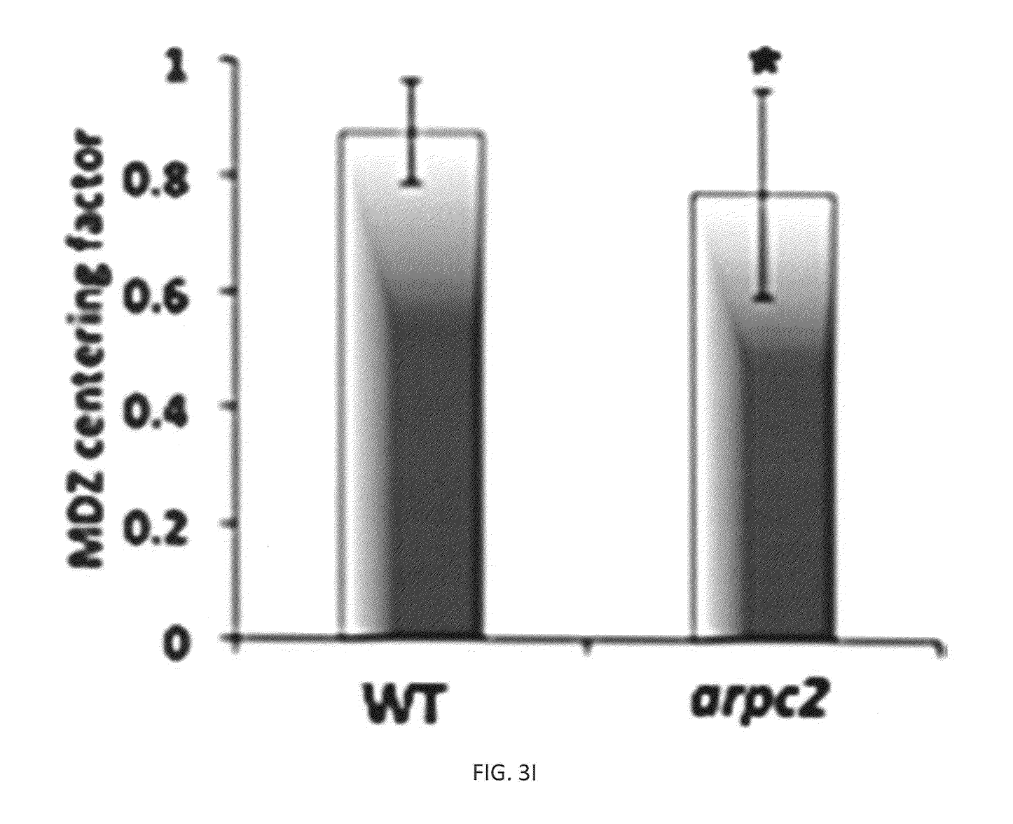

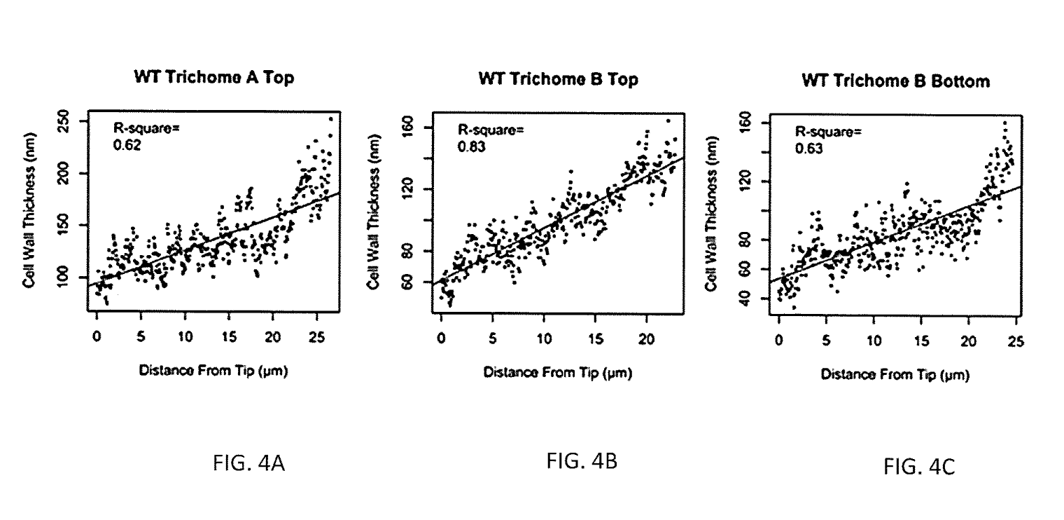

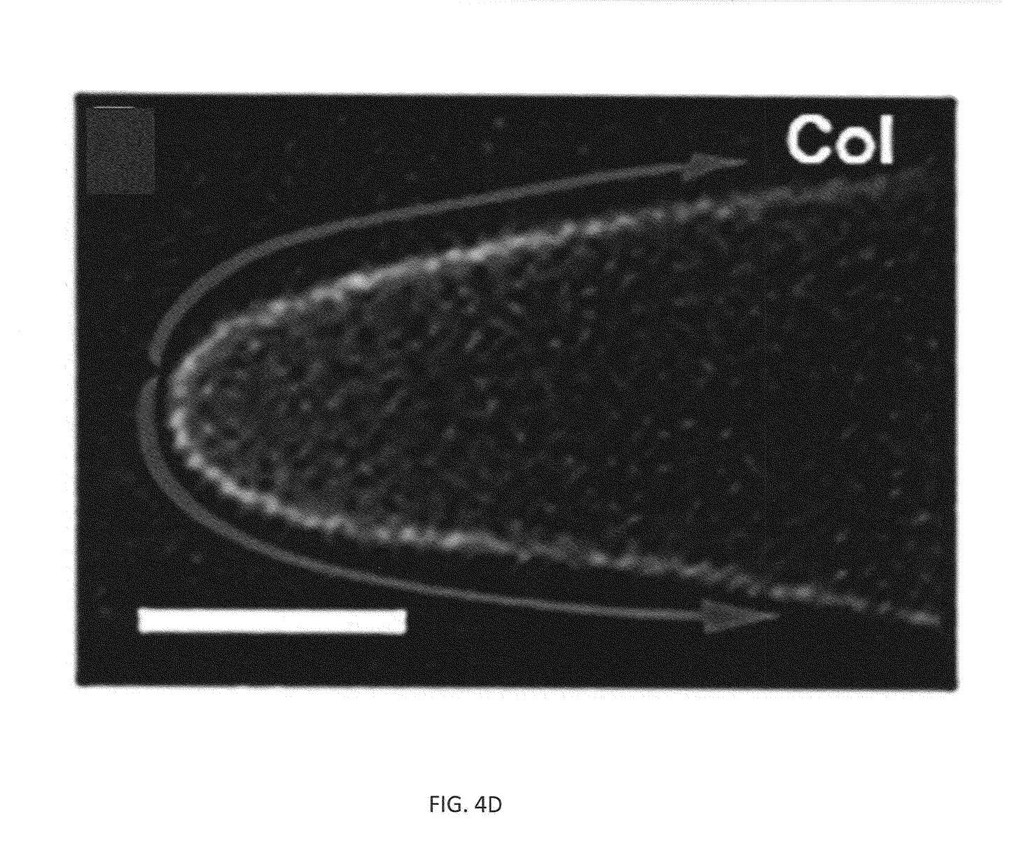

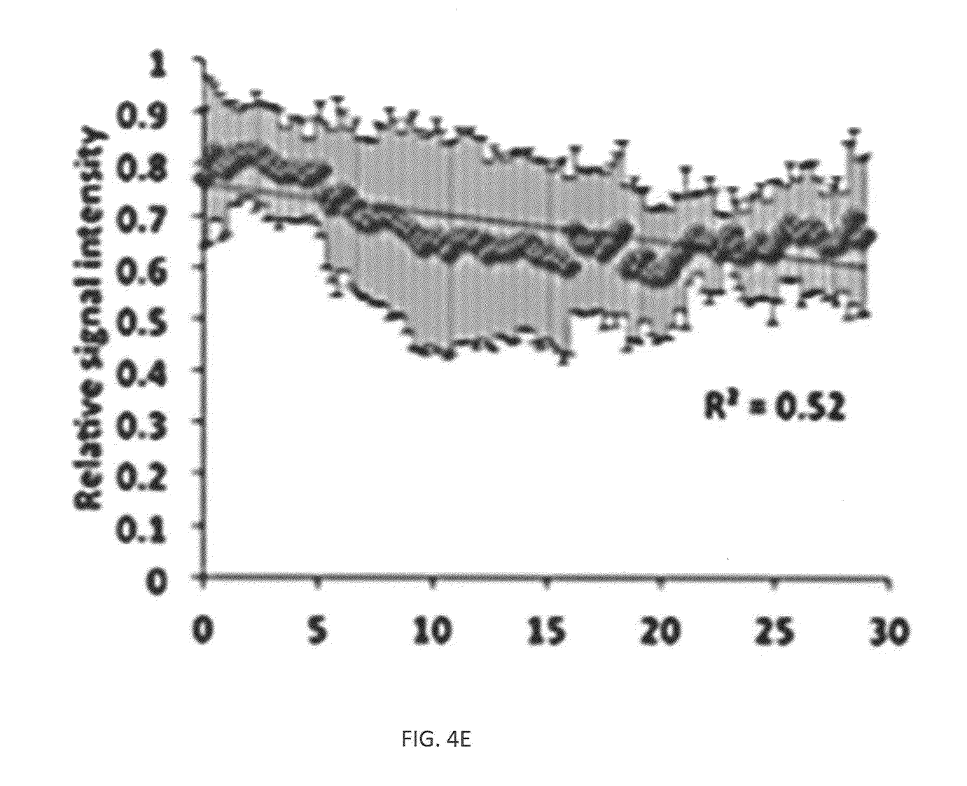

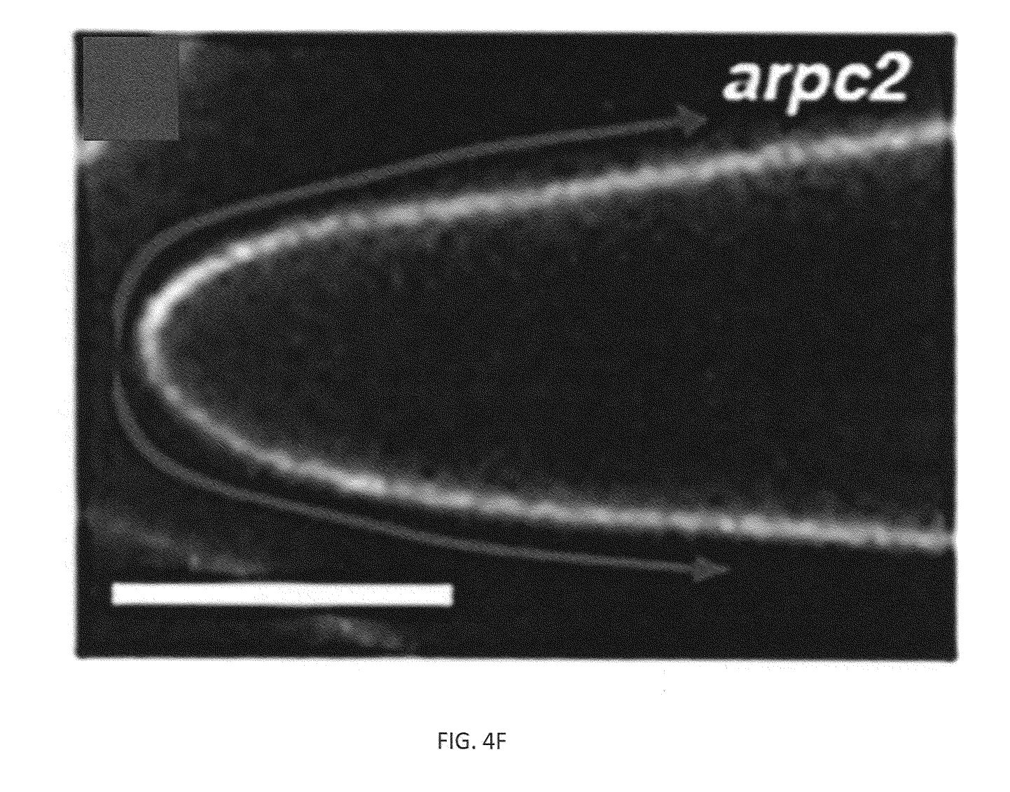

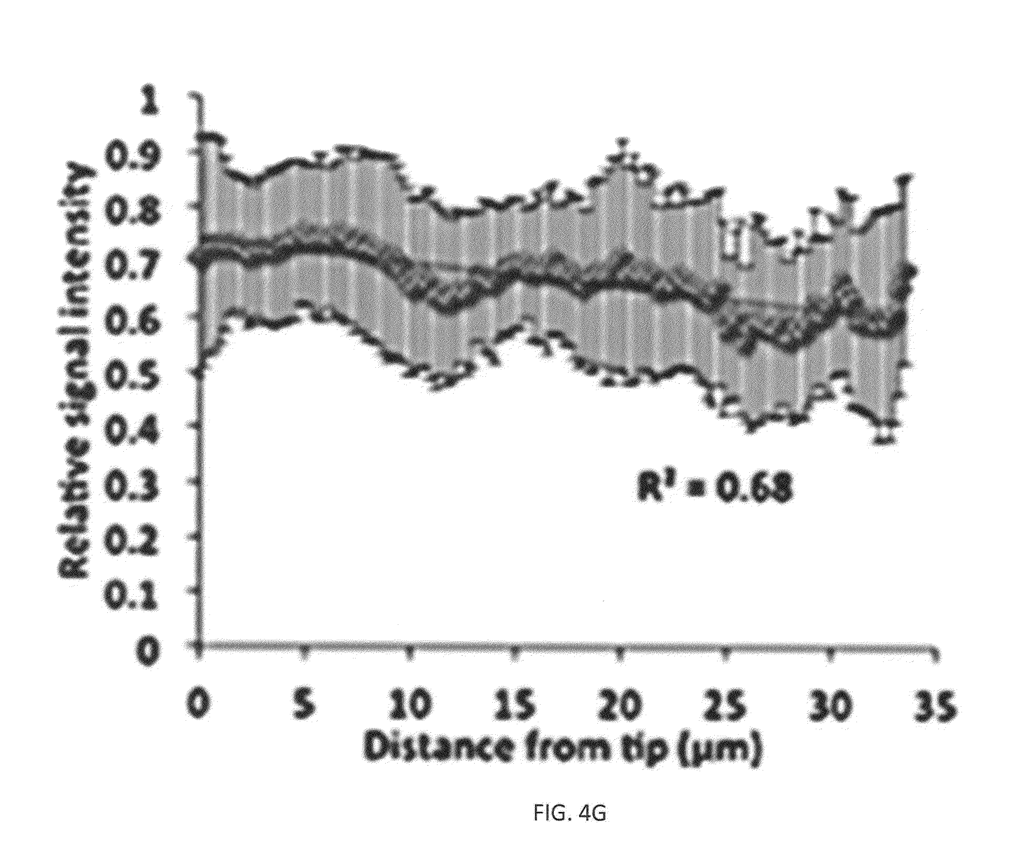

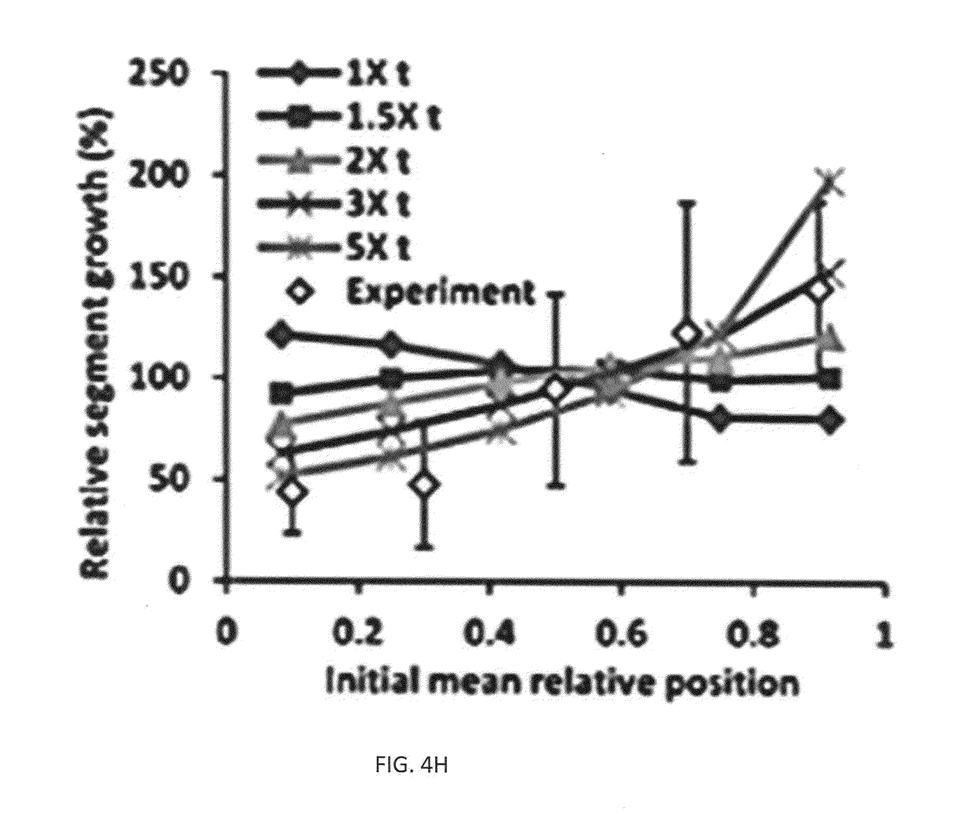

These FE simulations motivated experiments to quantify the predicted cell wall thickness gradient in trichome branches. First, wall thickness measurements were taken from TEM images of medial longitudinal sections through young trichome branches (FIG. 2G). Cell wall thickness was positively correlated with the distance from the branch tip (FIG. 2H). The magnitude of the tip to base wall thickness gradient for the wild type was 2.49, and similar values were obtained in replicate TEM analyses (FIGS. 4A-4C). To increase our sample size we used propidium iodide (PI) to quantify cell wall thickness variation. The normalized PI signal was also correlated with distance from the branch tip (FIG. 2I). The PI variation was not caused by cell geometry effects at the image plane, because the FM1-43 signal at the plasma membrane had a negative correlation with distance from the tip (FIGS. 4D-4G). These experimental data indicate that a wall thickness gradient exists with a sufficient magnitude to explain the observed proximo-distal strain gradients (FIG. 4H). Moreover, a combination of the measured thickness gradient with an elastic modulus gradient resulted in unrealistic growth predictions (FIG. 2F), thus excluding the simultaneous existence of a significant matrix modulus gradient. This differs from the tip-growth mechanism of pollen tubes, in which targeted secretion of methyl-esterified pectin at the apex locally decreases the matrix modulus. Treatment of growing leaves with pectinase released individual pavement and mesophyll cells, but did not cause any noticeable trichome swelling, suggesting further that pectin modification was not a critical component of shape control in this cell type.

The optimized FE model was used to simulate spatial stress and strain distributions of the young trichome branch (FIGS. 2J and 2K). The cell radius and wall thickness both contribute to the expected increase in the distribution of stress and strain. The sharp circular band of high stress near the branch apex is likely an overestimate due to the assumed abrupt transition from an anisotropic flank to isotropic tip in the FE simulations. The simulated strain distribution of the wall was consistent with the experimental bead labeling data. The model was also used to quantify the amount of new wall material added in order to maintain its thickness during cell expansion (FIG. 2L). It can be seen that there is a relatively shallow gradient of newly added material with higher values per element at the branch base. The simulation and wall marking data indicate that the mechanism of trichome morphogenesis bears little or no resemblance to tip growth, and instead is accomplished by highly regulated anisotropic diffuse growth in which spatial gradients in wall thickness and texture dictate the growth patterns.

Function and Formation of ARP2/3-Generated Actin Networks:

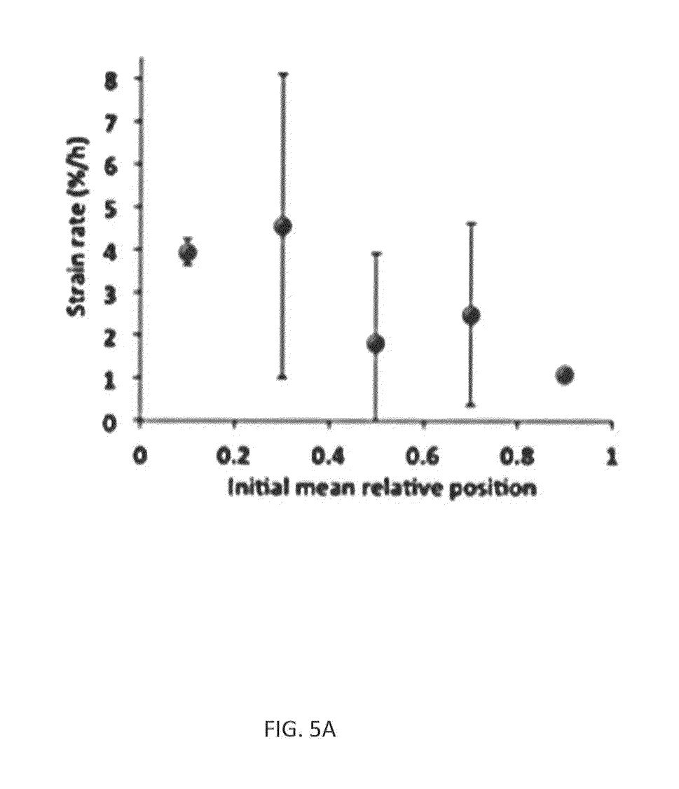

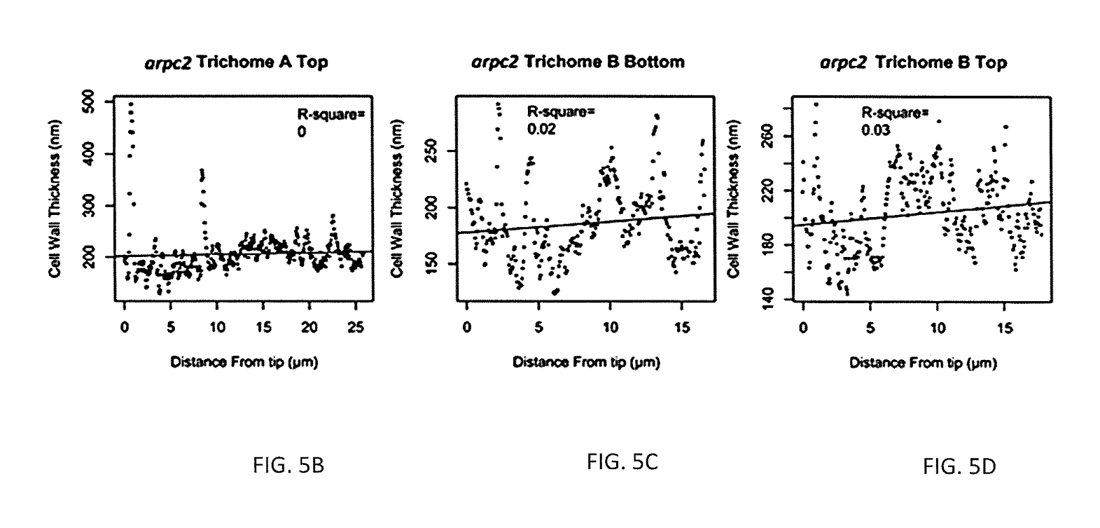

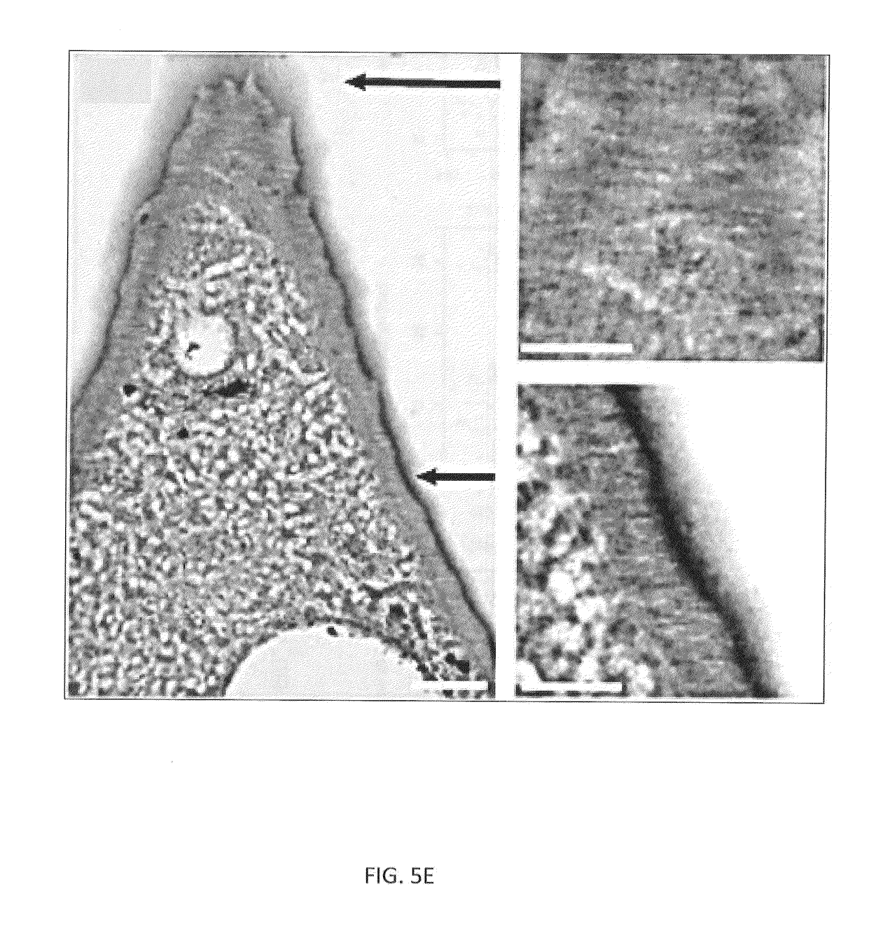

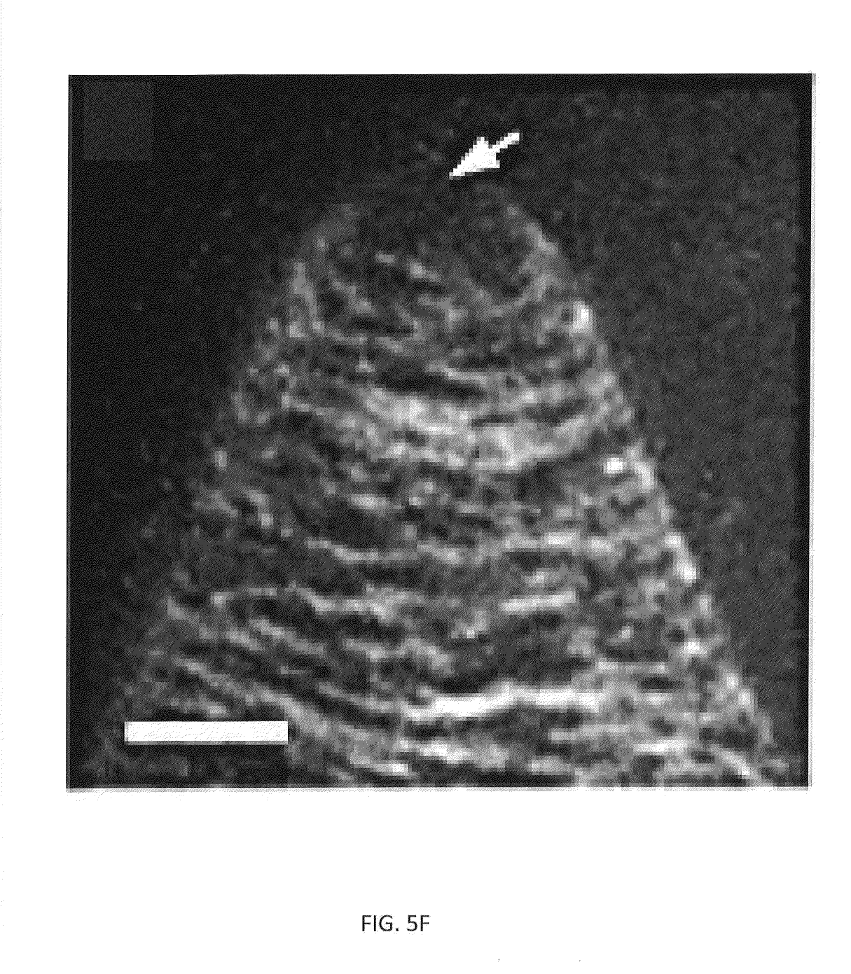

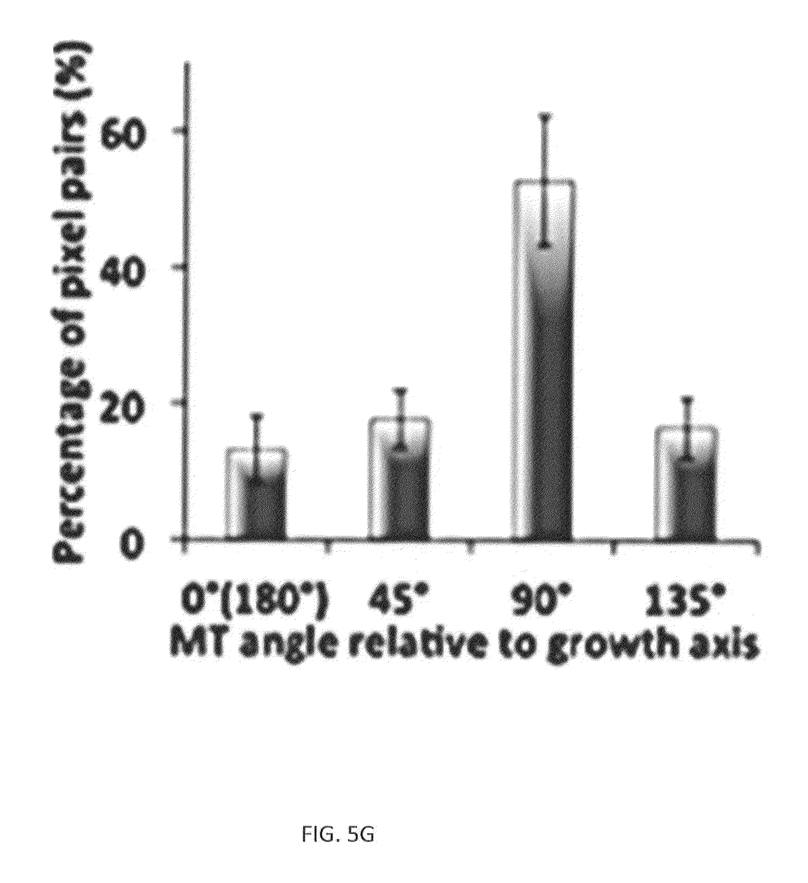

Microtubule-dependent microfibril patterning operates at cellular spatial scales to enable polarized branch elongation; however, the location and function of the most critical actin filament arrays are not known. In trichomes, ROP/RAC small GTPase signals generated by the ROPGEF SPIKE1 (SPK1) operate on the heteromeric W/SRC to positively regulate the actin filament nucleator ARP2/3. In arpc2/distorted2, the ARP2/3 complex is disassembled, and following branch initiation the branch base and stalk were swollen (FIG. 3A). The terminal arpc2 phenotype is a reduced branch length and severe cell swelling. Time-lapse analyses of arpc2 branches was technically challenging due cell twisting and swelling; however in some cases extended time-lapse was possible. We detected periods of little or no growth, punctuated by episodes of growth with highly variable rates (FIGS. 3B and 3C). We used visible cell wall protuberances to measure the local strain patterns of the fast growing arpc2 branch shown in FIG. 3B. The mutant branch did not have an increased strain rate in the distal regions of the branch (FIG. 5A).

We next tested for a cell wall thickness gradient in developing arpc2 branches. Although wall thickness had an increased local variability compared to the wild type, the mutant branches lacked a clear tip to base thickness gradient based on the regression analyses of the TEM and PI image data (FIGS. 3D-3F; FIGS. 5B-5D). The arpc2 cell walls had a significantly increased thickness of 202.4±11.8 nm compared to 109.7±22.7 for the wild type. The wall was cellulose-rich, as observed in glancing TEM sections of the wall, with obvious fibrillar texture that was transverse to the branch axis and parallel to cortical microtubules that were present in the mutant (FIGS. 5E-5G). Microtubule-based CESA insertion does not require an intact actin cytoskeleton. In aprc2 trichomes, growth and wall assembly are partially uncoupled, perhaps because cellulose synthesis is not coordinated with the delivery of wall matrix and/or wall loosening factors that enable normal wall strain behaviors. ARP2/3 had a second function at the branch tip based on the episodic and incomplete tip radius of curvature refinement detected in arpc2 (FIG. 3C). To test for an effect of arpc2 on the MDZ and the predicted TIZ, microtubule localization was analyzed in a population of arpc2 branches. Although the MDZ was clearly present in the mutant (FIG. 5F), its size was uncoupled from the geometry of the cell tip and its centering at the branch apex was less accurate compared to the wild type (FIGS. 3G-3I). Therefore, ARP2/3 is not required to generate an MDZ, but may function in concert with other ROP signaling pathways that locally destabilize microtubules, to position the microtubule free zone and modulate its size during tip refinement.

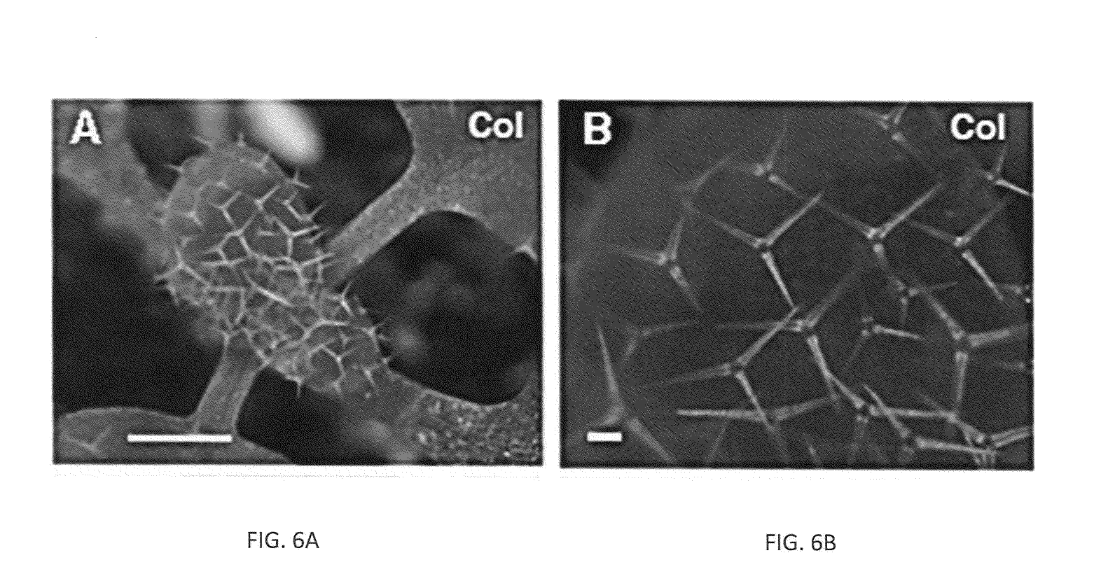

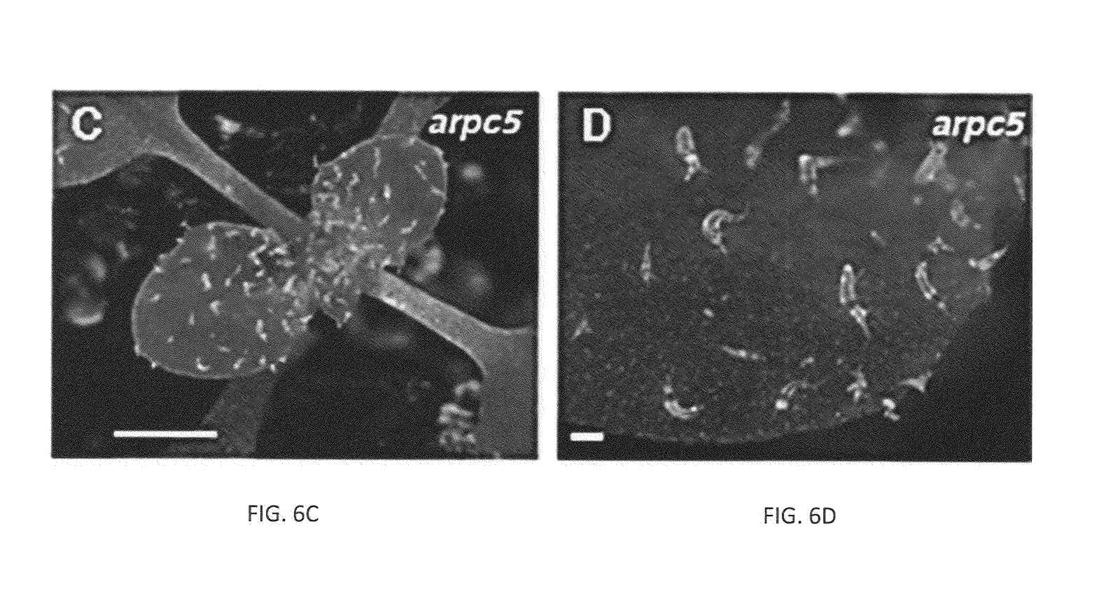

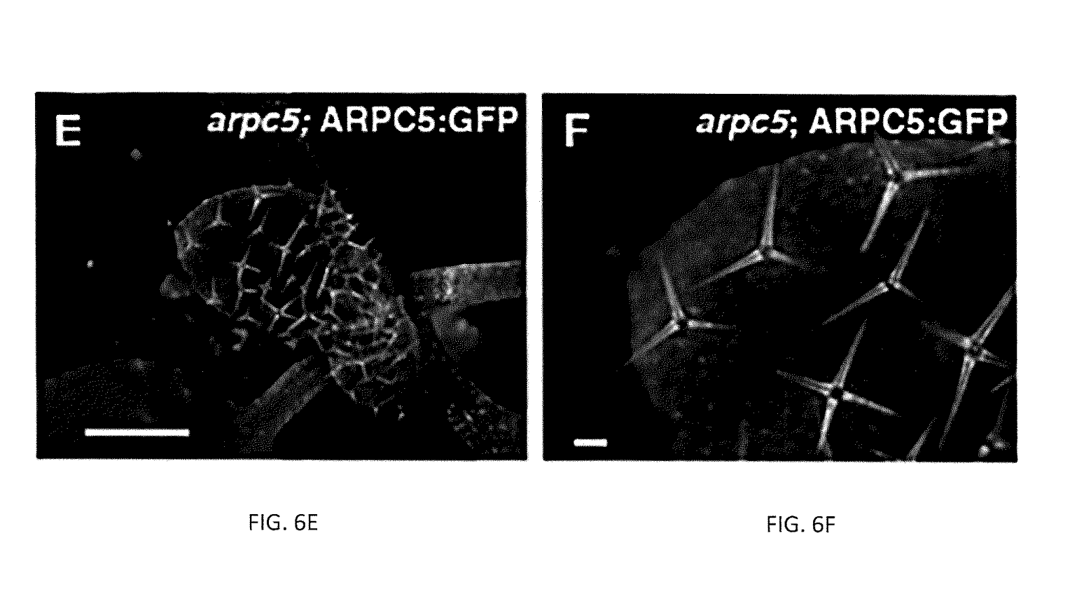

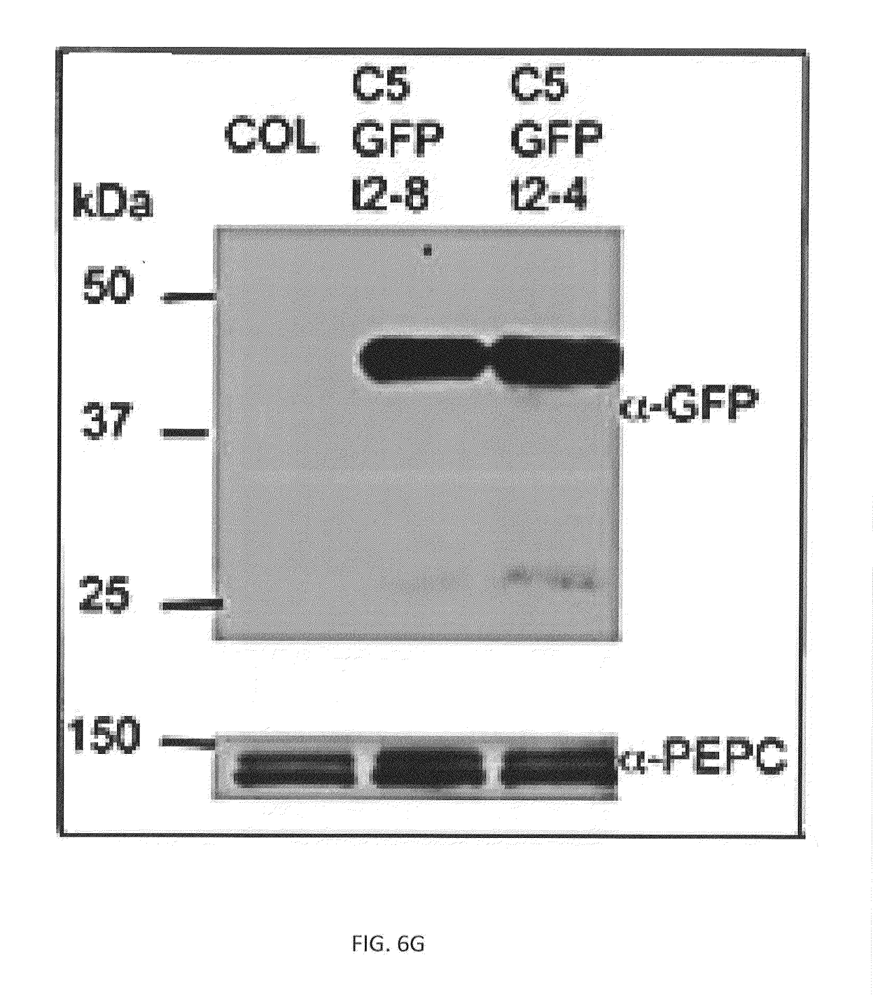

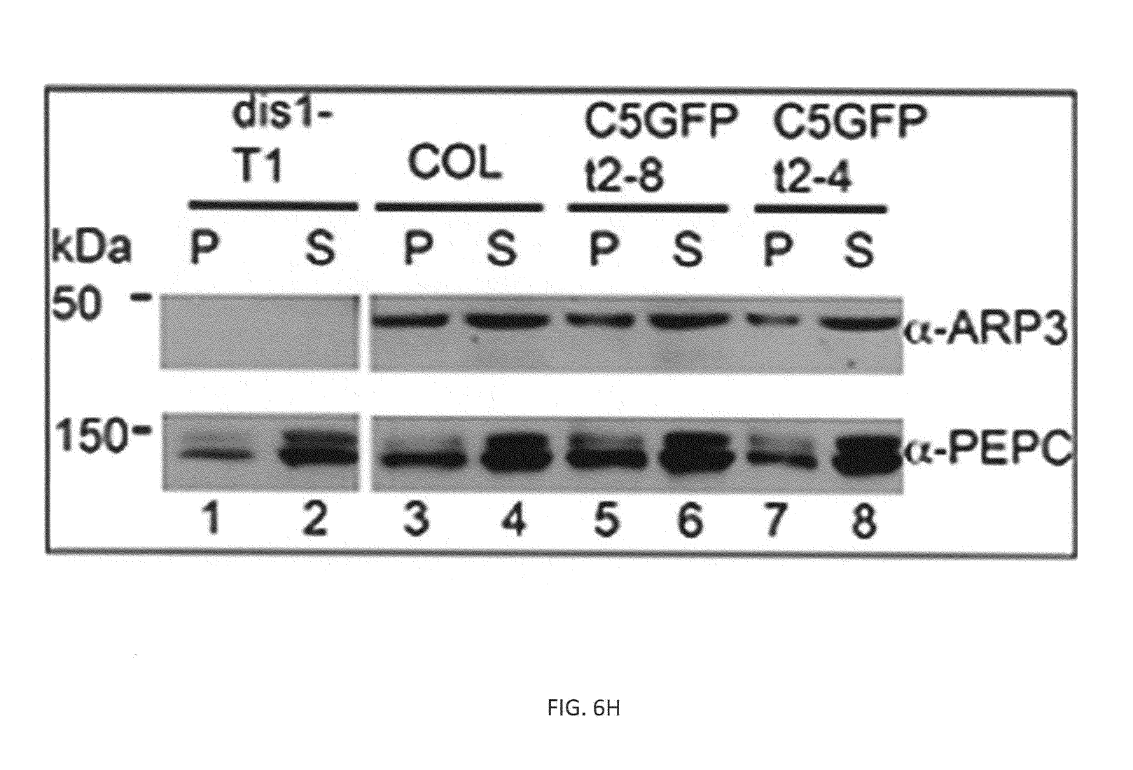

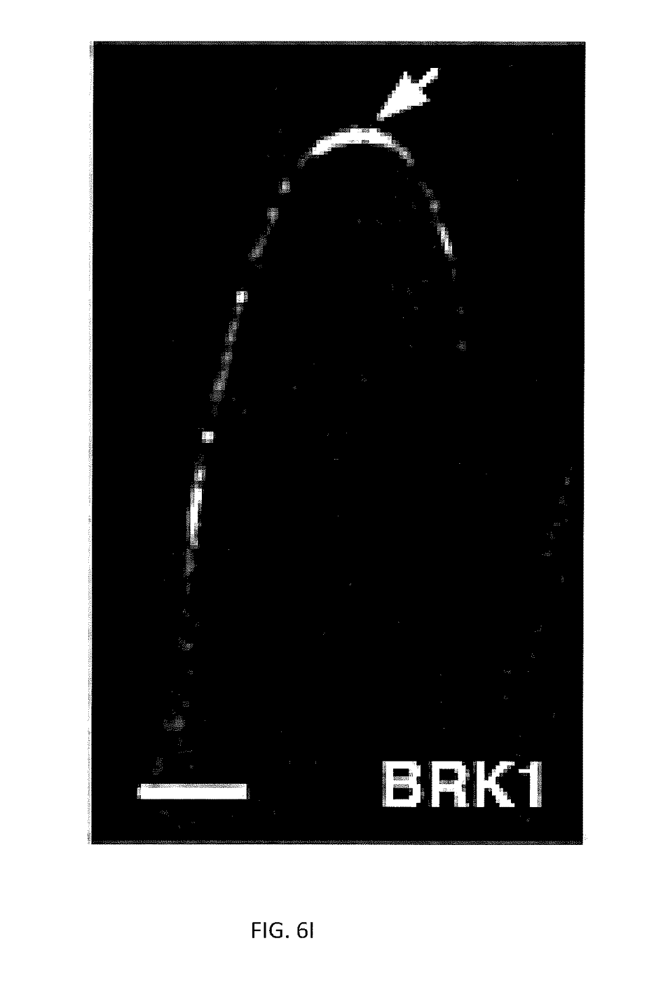

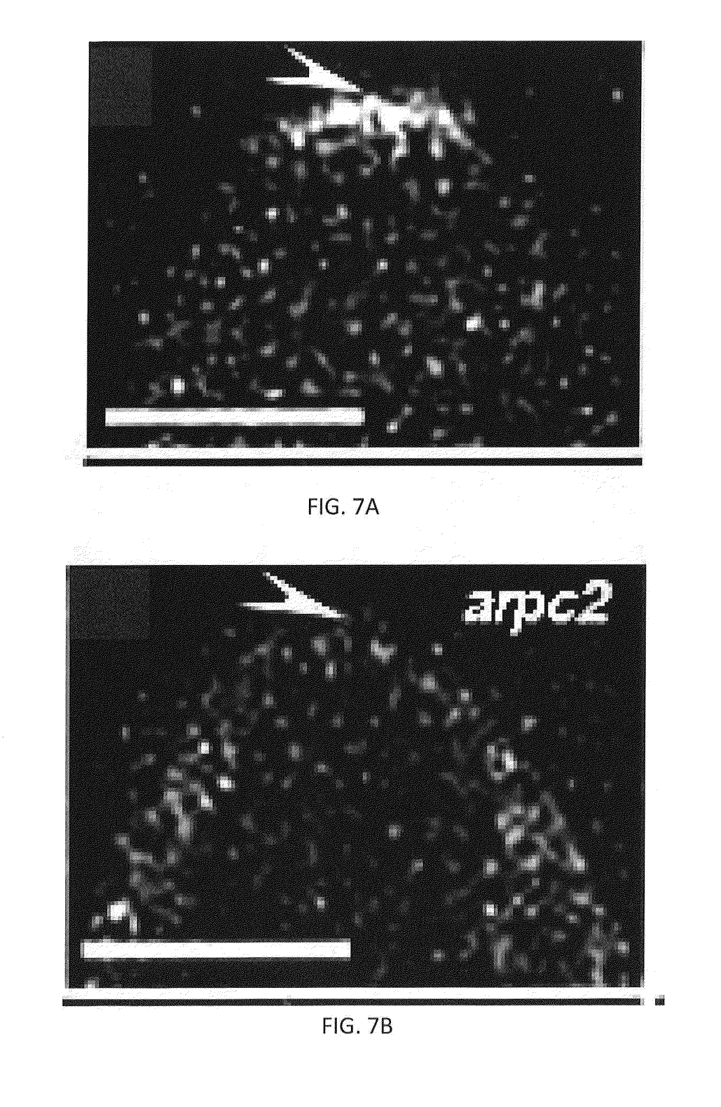

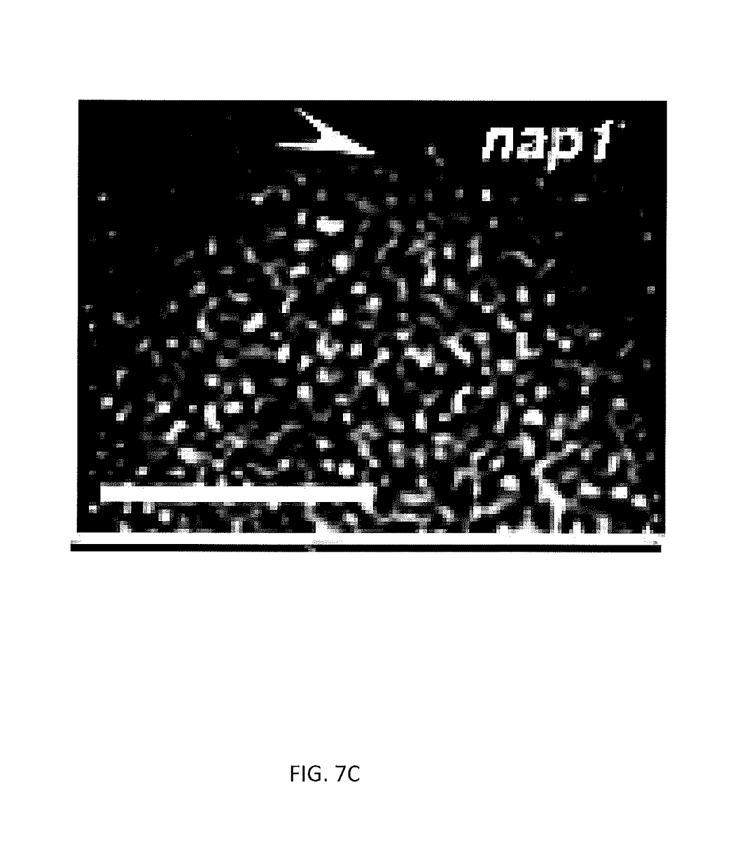

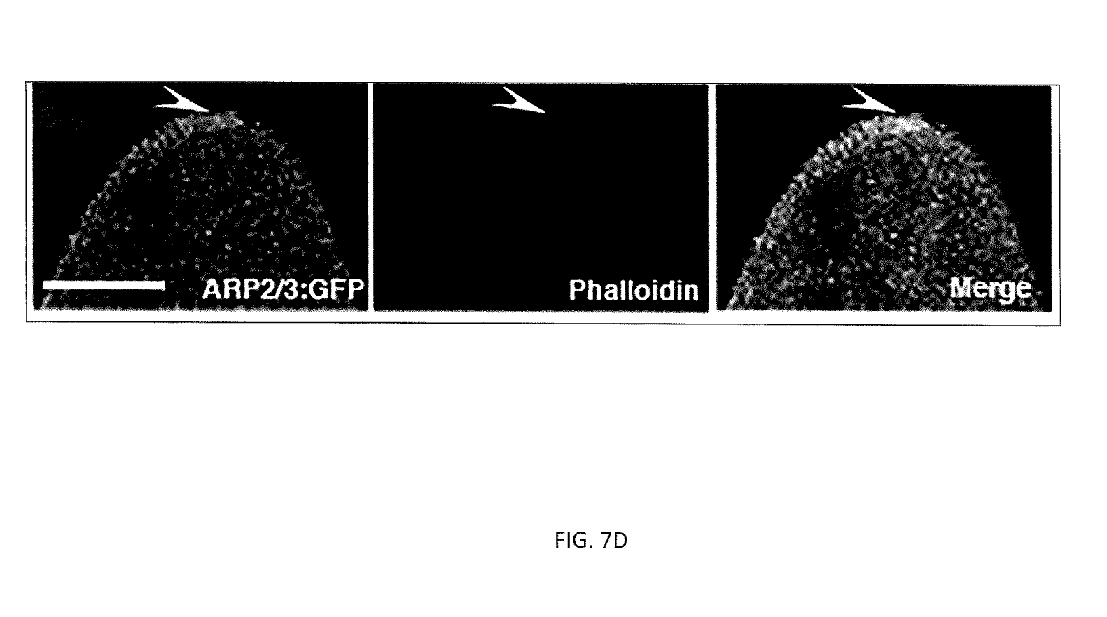

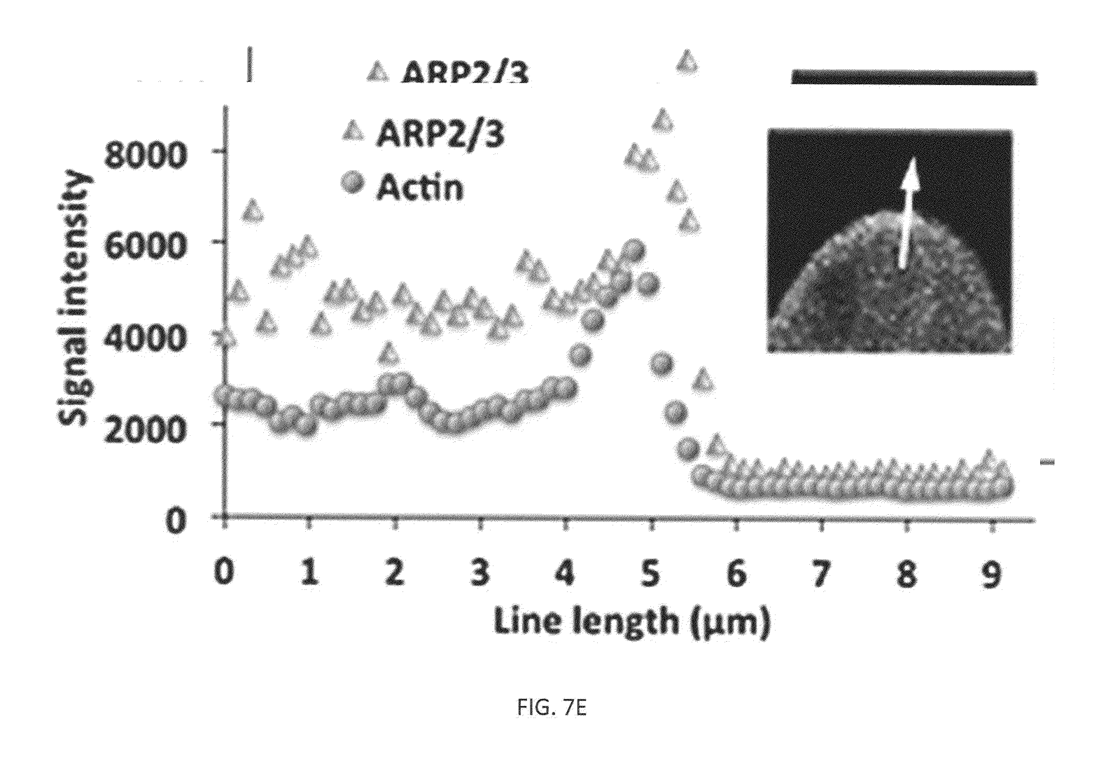

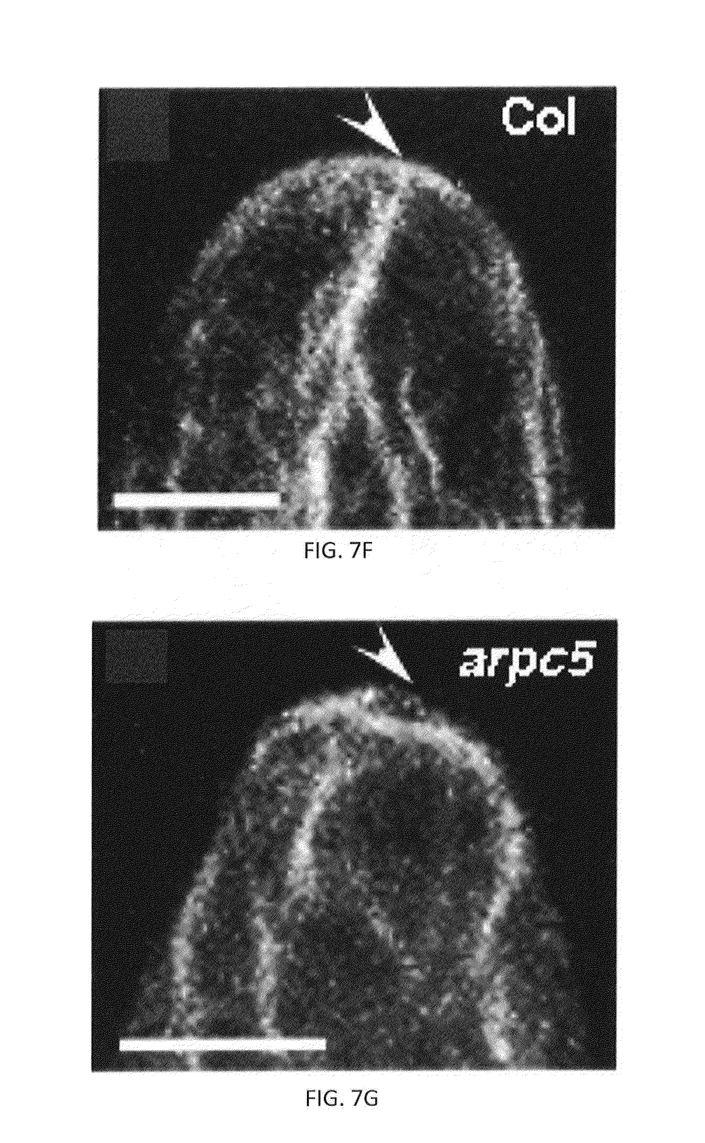

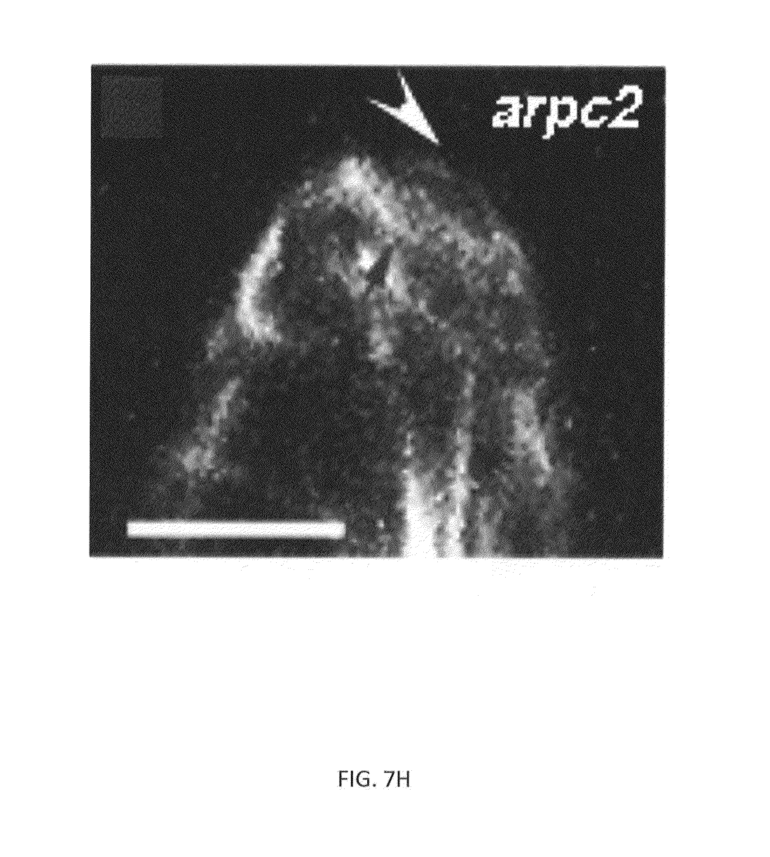

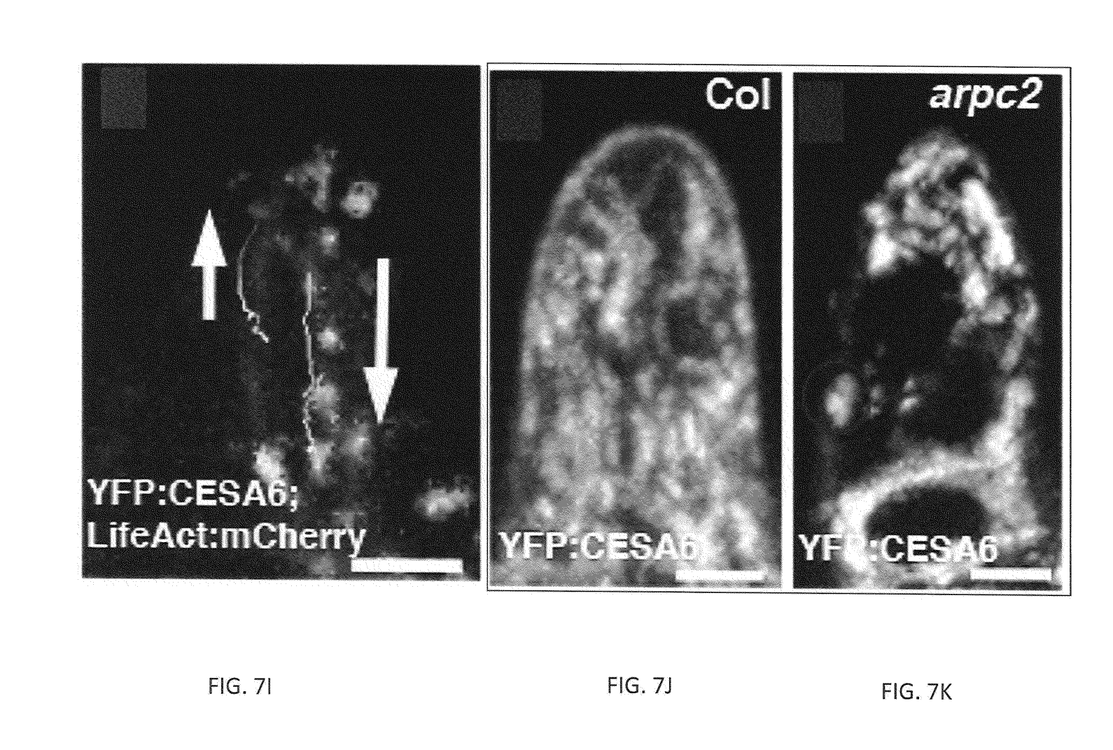

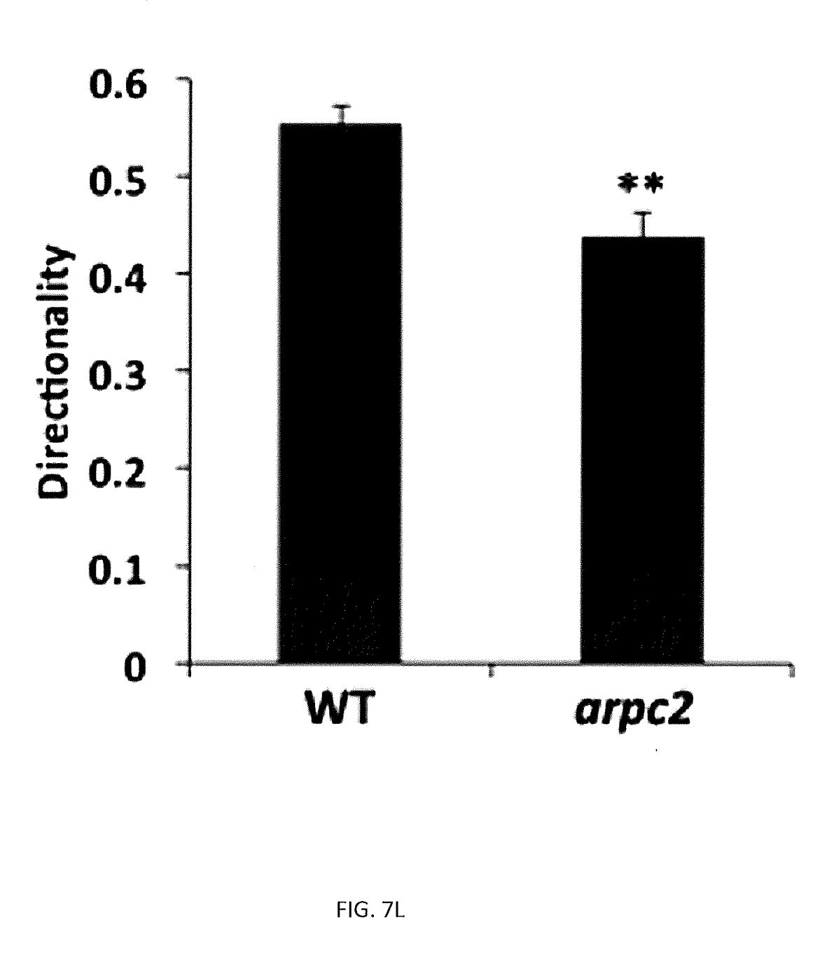

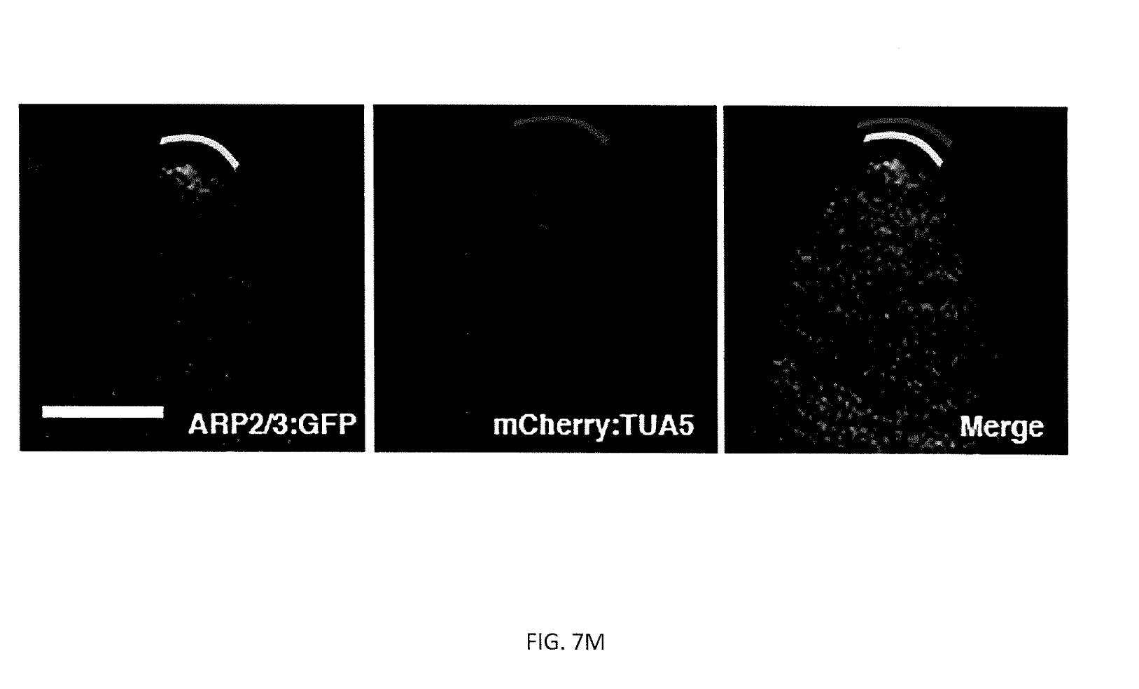

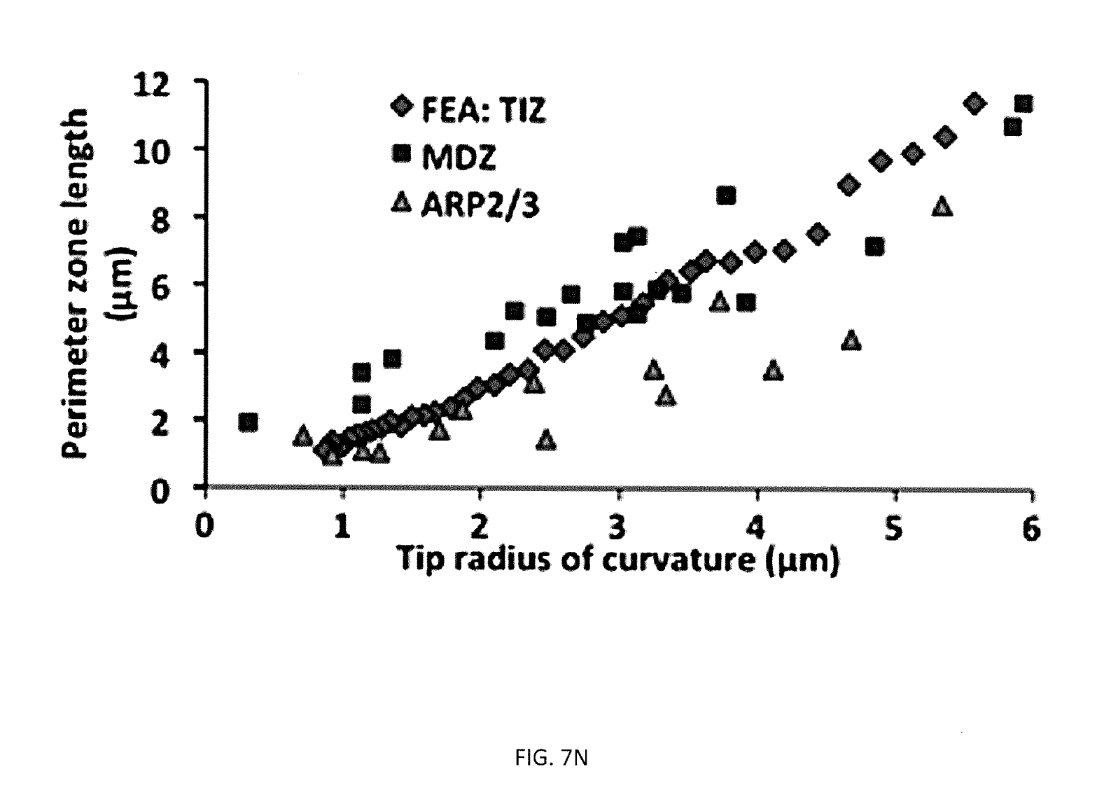

Our growth analyses suggested that ARP2/3 generates actin networks that have a global influence on the cell wall thickness gradient within a branch and a local influence on wall isotropy at the tip. However, the location of active ARP2/3 has not been determined in this, or any other plant cell type. We therefore created a fully functional GFP-tagged version of the ARPC5 subunit (FIGS. 6A-6H). GFP-tagged ARP2/3 was concentrated at the apex of young trichome branches (FIG. 7A). ARP2/3 and the known W/SRC subunit BRICK1 were clustered at the apex in 28% and 66% of the young trichome branches, respectively (Table 2, FIG. 6I). The significance of the reversible recruitment of ARP2/3 to the branch apex could not be analyzed due to photobleaching; however, when present at the tip ARP2/3 remained there for minutes. Tip localized ARP2/3 corresponded to an active pool of the complex, because this localization was eliminated in arpc2 (FIG. 7B) or in NAP1/GNARLED, which eliminates W/SRC-dependent positive regulation of ARP2/3 (FIG. 7C). Apical ARP2/3 was capable of full activation because it colocalized with cortical actin meshworks (FIGS. 7D and 7E) and the tiplocalized actin was ARP2/3-dependent (FIGS. 7F-7H). The presence of an apical actin meshwork was positively correlated with growth, because it was absent in arpc2, and only reduced in the arpc5 (Table 3), which has a more mild branch length phenotype compared with arpc2. The ARP2/3 generated cortical actin was related to core actin bundle positioning at the apex, because in 90% (n=20) of the cases in which the apical meshwork was present, core actin bundles terminated at or near it (FIGS. 7D and 7F). This result is consistent with previous reports of actin at the branch apex, and the known ARP2/3-dependence of actin bundle positioning within the core cytoplasm.

TABLE 2
Percentage of developing trichome branches showing tip signal.

| | | Tip signal | |
|---|---|---|---|
| Marker | Background | (%) | n |
| ARPC5:GFP | WT* | 28 | 123 |
| | arp2 | 7 | 99 |
| | arpc2 | 0 | 48 |
| | nap1 | 0 | 48 |
| | sra1 | 2 | 46 |
| BRK1:YFP | WT* | 66 | 55 |

*Rescued lines are shown as WT here.

TABLE 3
Percentage of Developing Trichome Branches
Showing Tip Actin Meshworks

| | Tip actin | | Mean branch length | |
|---|---|---|---|---|
| Genotypes | (%) | n | (μm) | Ref. |
| WT | 60 | 63 | 190 | (19) |
| arpc5 | 22 | 68 | 70 | (26) |
| arpc2 | 0 | 61 | 47 | (19) |

Based on the distorted trichome phenotype of MYOSIN XI mutants, acto-myosindependent organelle transport is a key component of growth control. Our time-lapse analyses of Golgi transport and actin bundle localization in wild-type trichomes did not detect an obviously organized flow pattern which is typical of tip-growing cells; however, there was evidence for directional movement as a subset of Golgi staggered along actin bundle tracks (FIG. 7I). Frequent pauses, bidirectional flow along bundles with differing polarities, and exchange between the cortical and core cytoplasm efficiently circulated the Golgi throughout the branch as judged by projections of time-lapse images (FIG. 7J). Large numbers of motile Golgi were present in young arpc2 branches; however, the directionality of their movement was reduced (FIG. 7L), and projections of time series images showed that Golgi motility was restricted to narrow trafficking lanes that were randomly positioned compared to the wild type (FIGS. 7K). Defects in the actin cytoskeleton organization have been linked to wall thickness and cell shape defects in hypocotyl epidermal cells. Given the cell wall thickness defects in arpc2, ARP2/3 appears to operate at cellular scales by organizing an actin transport network that distributes secretory organelles throughout the cytoplasm so that wall assembly and growth are properly coupled. Certainly, additional levels of control at the plasma membrane, such as exocyst-dependent vesicle fusion, may enable the cell to more precisely balance the secretion of matrix components with cellulose synthesis during morphogenesis.

ARP2/3-generated apical actin meshworks may also have a more local function to position the MDZ during growth. We found that the size of the ARP2/3-positive apical domain was strongly correlated with the tip geometry, and based on 2-color live cell imaging, it was always positioned within the MDZ (FIGS. 7M and 7N). An importance for modulation of the size of the TIZ was also demonstrated with the FE model, because an improved fit with the experimental data was obtained when the regression lines from the MDZ and ARP2/3 localization data were used to couple the size of the TIZ and the radius of curvature during a simulation (FIG. 7O, Table 4). ARP2/3-mediated modulation of the TIZ is a plausible mechanism to achieve tip radius refinement, because a perfect fit between the simulated and observed tip radius refinement could be generated by optimizing the size of the TIZ throughout the simulation (FIG. 7O).

TABLE 4
Fit of tip radius of curvature versus branch length for differently
modulated TIZ sizes during growth in terms of root-mean-square
deviation (RMSD). TIZ size was modulated during growth based
on the regression lines from ARP2/3 or MDZ localization data
as well as held constant. MDZ-modulated TIZ variation during
growth demonstrated the best fit.

| | RMSD |
|---|---|
| Simulation TIZ | (μm) |
| Experimental Fit | 0.4080 |
| TIZ = ARP2/3 size | 0.7356 |
| TIZ = MDZ size | 0.6056 |
| TIZ = 0.25 μm | 1.5594 |
| TIZ = 0.5 μm | 1.1566 |
| TIZ = 1 μm | 0.727 |
| TIZ = 1.5 μm | 0.878 |
| TIZ = 2 μm | 1.3629 |

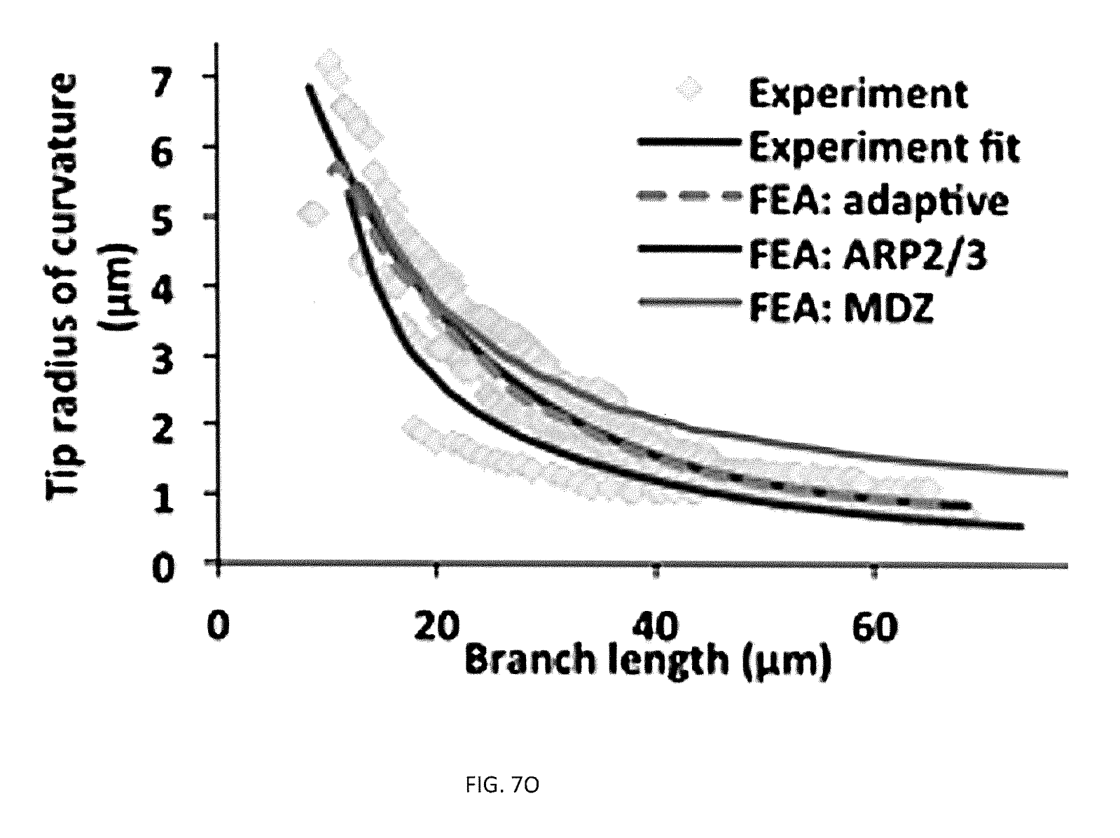

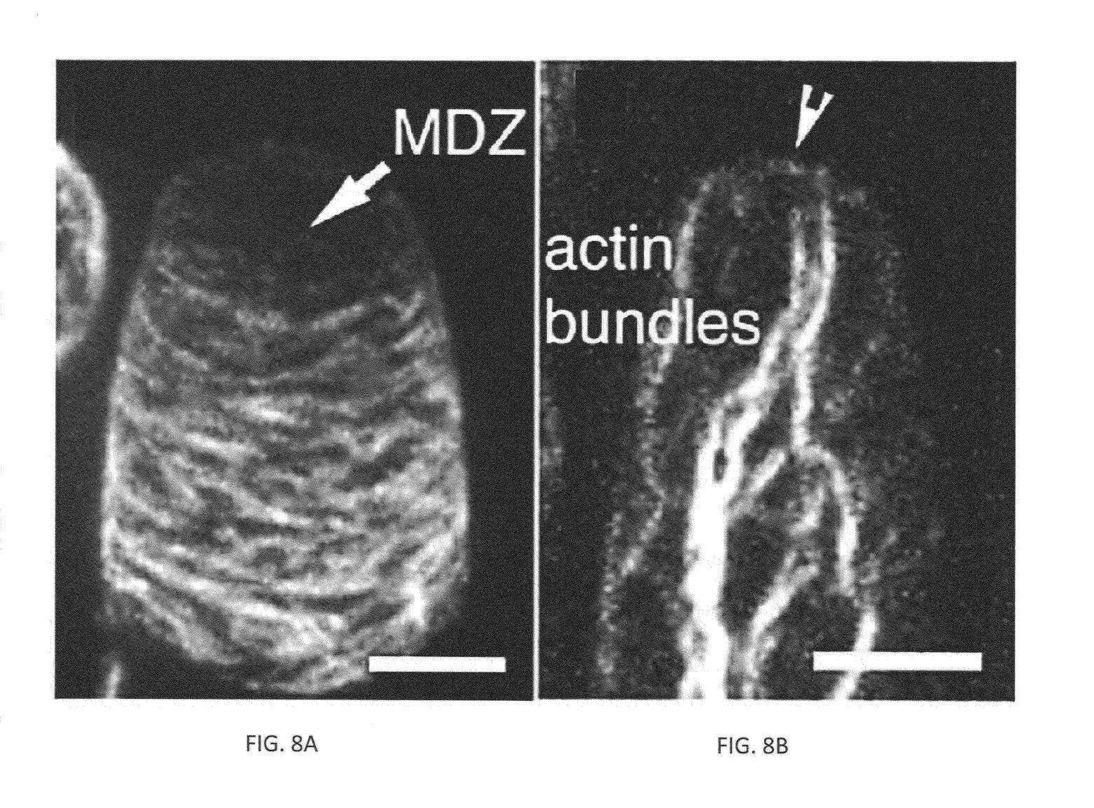

As further demonstrative that a microtubule-depletion exists in the tip of cotton fibers and that the arrangement the actin network is similar to what is seen in Arabidopsis, referring to FIGS. 8A and 8B, it is herein shown that accepted methods for cell actin and microbtubule labeling in Arabidopsis trichomes are effective in cotton fibers even at the earliest developmental stages that are poorly characterized. The apical microtubule-depletion zone and apical actin meshworks that are central to tip refinement in leaf hairsa are also present in cotton fibers (FIGS. 8A and 8B). Along similar lines, the FE models and their biomechanical assays of plant cell walls can be translated easily to cultured cotton fibers.

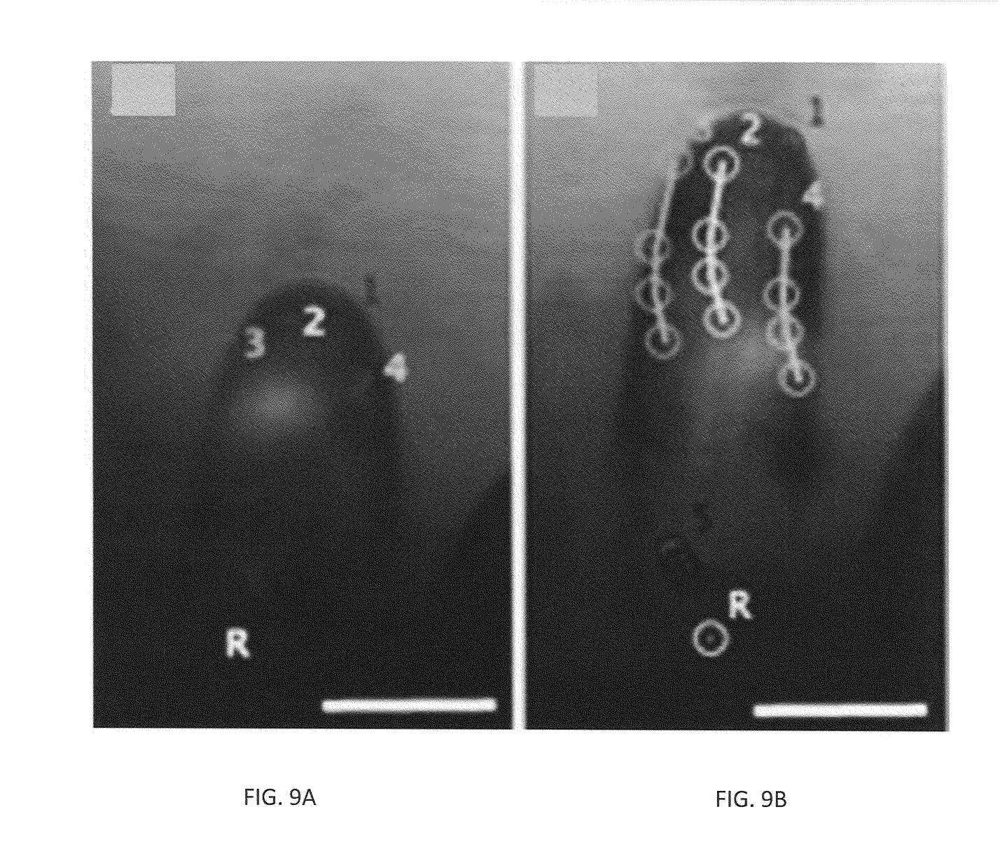

Diffuse growth is involved in cotton fiber elongation, but the potential involvement of tip growth is frequently discussed. Time lapse analysis and wall marking experiments are needed to quantitate the patterns of diffuse growth94 and test for a tip growth component. Bead labeling methods have been developed for Arabidopsis trichomes that allow quantification of local strain rates and parameterize realistic FE models of polarized diffuse growth. Identical methods can be used in combination with known ovule culture methods. Cultured fibers were labeled with red-fluorescent sulfated polystyrene beads as fiducial marks (FIG. 9A), that were tracked over time to monitor the patterns of cell wall strain (FIG. 9B). Strain rates were ˜5% hr⁻¹ along the cell flanks; however beads at the apex were not displaced relative to the cell tip. This result is inconsistent with a tip growth mechanism, because if tip growth occurred, the beads would be quickly displaced from the apex to the flank as new cell wall material is added exclusively at the tip. These bead-tracking data demonstrate our ability to conduct subcellular wall strain analyses that are compatible with simultaneous imaging of GFP-tagged proteins.

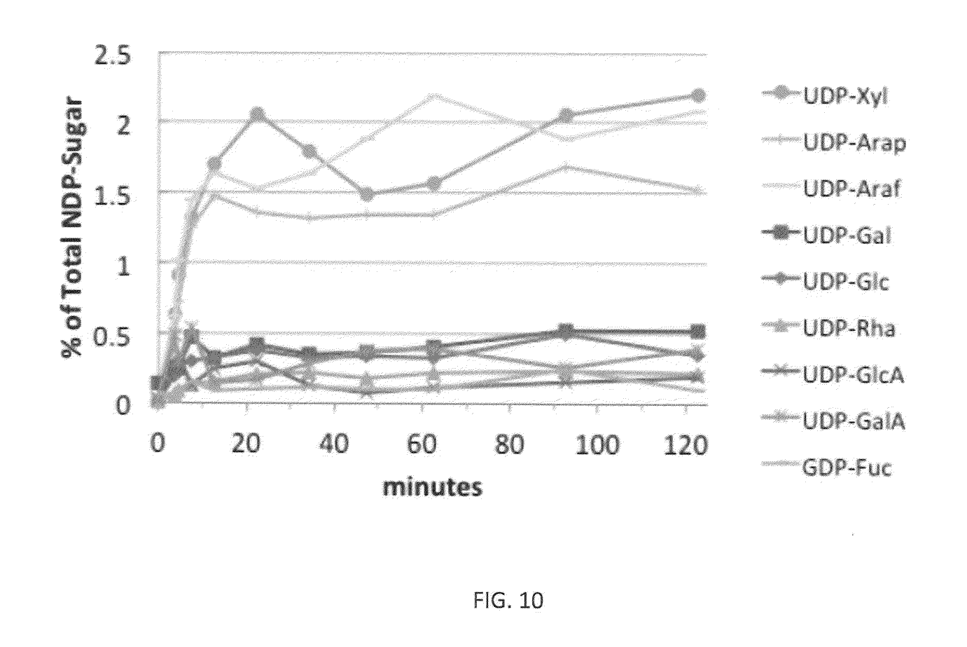

The ovule culture system is a valid system to analyze cell wall composition as a function of development. Such methods can be applied to the cotton fiber system. The flux of carbon between different cell wall pools and central carbohydrate metabolic pathways can be examined. Such methods involved pulse chase labeling of sugars with stable isotopes and kinetic modeling of the sugar into different metabolic pools. ¹³C-xylose fed to cell cultures moves into different nucleotide sugars through different pathways (FIG. 10). Nucleotide sugars are the key precursors of cell wall biosynthesis.

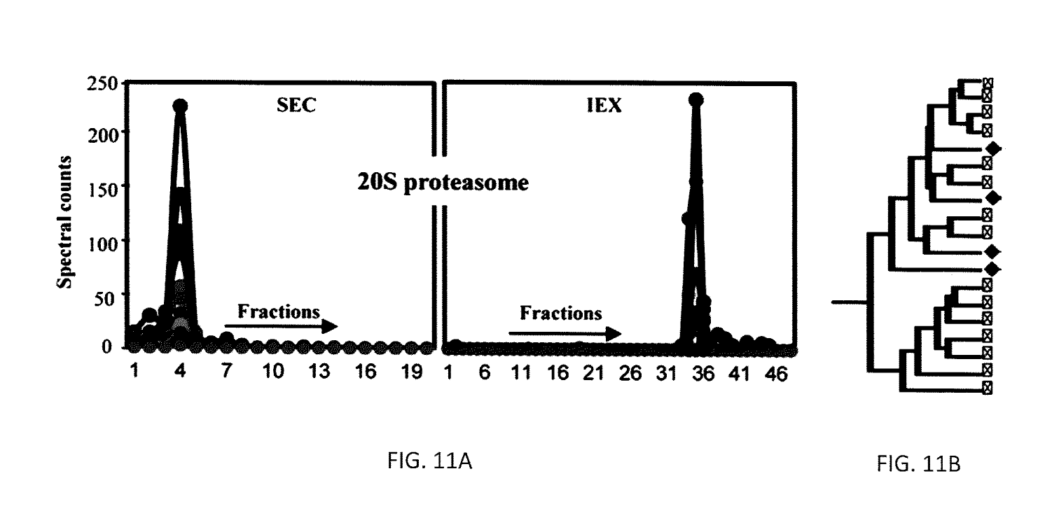

Proteomic data sets on the composition and localization of protein complexes can be useful as well. The feasibility of high-throughput protein quantification and detection of protein complexes in the Arabidopsis system is confirmed. Additional unpublished data from Arabidopsis shows we can predict protein complex composition with reasonable accuracy by clustering the abundance profiles of 1593 reproducibly detected endogenous proteins that were separated by size (size exclusion chromatography, SEC) and the profiles of the same input material separated by charge (ion-exchange chromatography, IEX). FIG. 11A shows the abundance profiles of 16 of the 28 known 20S proteasome subunits that we detected. Upon clustering analysis of the complete dataset, 15 of the proteasome subunits are correctly predicted to form a complex (FIG. 11B).

Similar experiments are feasible in cotton. From 400 μg of soluble protein obtained from 13 DPA G. hirsutum fibers (only 2 bolls), we quantified over 1300 unique soluble proteins across 23 SEC fractions. 640 of these proteins, including dozens of cytoskeletal (27 cytoskeletal proteins), cell wall proteins (for example, AGPs, RGP, Glycosyl tranferases), and 19 enzymes in nucleotide sugar metabolism that are directly relevant, were predicted to be oligomeric based on their measured apparent mass. These data are important because they show the resolving power of our protein complex characterization method and the feasibility of applying these techniques to cotton.

Those skilled in the art will recognize that numerous modifications can be made to the specific implementations described above. The implementations should not be limited to the particular limitations described. Other implementations may be possible. In addition, all references cited herein are indicative of the level of skill in the art and are hereby incorporated by reference in their entirety.

### REFERENCES

1. R. Mauricio, Costs of resistance to natural enemies in field populations of the annual plant Arabidopsis thaliana. Am. Nat. 151, 20 (1998).
2. S. C. Tiwari, T. A. Wilkins, Cotton (Gossypium hirsutum) seed trichomes expand via diffuse growing mechanism. Can. J. Bot. 73, 746 (1995).
3. D. B. Szymanski, M. D. Marks, S. M. Wick, Organized F-actin is essential for normal trichome morphogenesis in Arabidopsis. Plant Cell 11, 2331 (1999).
4. J. Mathur, P. Spielhofer, B. Kost, N. Chua, The actin cytoskeleton is required to elaborate and maintain spatial patterning during trichome cell morphogenesis in Arabidopsis thaliana. Development 126, 5559 (1999).
5. T. I. Baskin, Anisotropic expansion of the plant cell wall. Annu. Rev. Cell Dev. Biol. 21, 203 (2005).
6. A. R. Paredez, C. R. Somerville, D. W. Ehrhardt, Visualization of cellulose synthase demonstrates functional association with microtubules. Science 312, 1491 (2006).
7. C. Zhang et al., Arabidopsis SCARs function interchangeably to meet actin-related protein 2/3 activation thresholds during morphogenesis. Plant Cell 20, 995 (2008).
8. J. Mathur et al., Arabidopsis CROOKED encodes for the smallest subunit of the ARP2/3 complex and controls cell shape by region specific fine F-actin formation. Development 130, 3137 (2003).
9. P. Fayant et al., Finite element model of polar growth in pollen tubes. Plant Cell 22, 2579 (2010).
10. B. Schwab et al., Regulation of cell expansion by the DISTORTED genes in Arabidopsis thaliana: actin controls the spatial organization of microtubules. Mol. Genet. Genome 269, 350 (2003).
11. M. Fujita et al., The anisotropyl D604N mutation in the Arabidopsis cellulose synthasel catalytic domain reduces cell wall crystallinity and the velocity of cellulose synthase complexes. Plant physiol. 162, 74 (2013).
12. T. C. Gasser, R. W. Ogden, G. A. Holzapfel, Hyperelastic modelling of arterial layers with distributed collagen fibre orientations. Journal of the Royal Society, Interface/the Royal Society 3, 15 (2006).
13. R. Huang, A. A. Becker, I. A. Jones, Modelling cell wall growth using a fibre-reinforced hyperelastic-viscoplastic constitutive law. J. Mech. Phys. Solids 60, 750 (2012).
14. S. T. McKenna et al., Exocytosis precedes and predicts the increase in growth in oscillating pollen tubes. Plant Cell 21, 3026 (2009).
15. S. Timoshenko, Strength of materials. (New York, 1930).
16. D. Basu, J. Le, T. Zakharova, E. L. Mallery, D. B. Szymanski, A SPIKE1 signaling complex controls actin-dependent cell morphogenesis through the heteromeric WAVE and ARP2/3 complexes. Proc. Natl. Acad. Sci. U.S.A. 105, 4044 (2008).
17. D. Basu, S. E. El-Assal, J. Le, E. L. Mallery, D. B. Szymanski, Interchangeable functions of Arabidopsis PIROGI and the human WAVE complex subunit SRA1 during leaf epidermal development. Development 131, 4345 (2004).
18. M. J. Deeks, D. Kaloriti, B. Davies, R. Malho, P. J. Hussey, Arabidopsis NAP1 is essential for ARP2/3-dependent trichome morphogenesis. Curr. Biol. 14, 1410 (2004).
19. D. Basu et al., DISTORTED3/SCAR2 is a putative Arabidopsis WAVE complex subunit that activates the Arp2/3 complex and is required for epidermal morphogenesis. Plant Cell 17, 502 (2005).
20. S. O. Kotchoni et al., The association of the Arabidopsis actin-related protein (ARP) 2/3 complex with cell membranes is linked to its assembly status, but not its activation. Plant Physiol. 151, 2095 (2009).
21. S. E. El-Assal, J. Le, D. Basu, E. L. Mallery, D. B. Szymanski, DISTORTED2 encodes an ARPC2 subunit of the putative Arabidopsis ARP2/3 complex. Plant J. 38, 526 (2004).
22. A. Sampathkumar et al., Patterning and lifetime of plasma membrane-localized cellulose synthase is dependent on actin organization in Arabidopsis interphase cells. Plant physiol. 162, 675 (2013).
23. L. Lu, Y.-R. J. Lee, R. Pan, J. N. Maloof, B. Liu, An internal motor kinesin is associated with the golgi apparatus and plays a role in trichome morphogenesis in Arabidopsis. Mol. Biol. Cell 16, 811 (2005).
24. Y. Oda, H. Fukuda, Initiation of cell wall pattern by a Rho- and microtubule-driven symmetry breaking. Science 337, 1333 (2012).
25. J. Le, E. L. Mallery, C. Zhang, S. Brankle, D. B. Szymanski, Arabidopsis BRICK1/HSPC300 is an essential WAVE-complex subunit that selectively stabilizes the Arp2/3 activator SCAR2. Curr. Biol. 16, 895 (2006).
26. S. Djakovic, J. Dyachok, M. Burke, M. J. Frank, L. G. Smith, BRICK1/HSPC300 functions with SCAR and the ARP2/3 complex to regulate epidermal cell shape in Arabidopsis. Development 133, 1091 (2006).
27. J. Dyachok et al., Plasma membrane-associated SCAR complex subunits promote cortical F-actin accumulation and normal growth characteristics in Arabidopsis roots. i Mol. Plant 1, 990 (2008).
28. S. E. El-Assal, J. Le, D. Basu, E. L. Mallery, D. B. Szymanski, Arabidopsis GNARLED encodes a NAP125 homologue that positively regulates ARP2/3. Curr. Biol. 14, 1405 (2004).
29. J. Le, S. E. El-Assal, D. Basu, M. E. Saad, D. B. Szymanski, Requirements for Arabidopsis ATARP2 and ATARP3 during epidermal development. Curr. Biol. 13, 1341 (Aug. 5, 2003).
30. X. Zhang, J. Dyachok, S. Krishnakumar, L. G. Smith, D. G. Oppenheimer, IRREGULAR

TRICHOME BRANCH1 in Arabidopsis encodes a plant homolog of the actin-related protein2/3 complex activator Scar/WAVE that regulates actin and microtubule organization. Plant Cell 17, 2314 (2005).
31. A. Sambade, K. Findlay, A. R. Schaffner, C. W. Lloyd, H. Buschmann, Actin-dependent and -independent functions of cortical microtubules in the differentiation of Arabidopsis leaf trichomes. Plant Cell 26, 1629 (2014).
32. E. L. Ojangu et al., Myosins XI-K, XI-1, and XI-2 are required for development of pavement cells, trichomes, and stigmatic papillae in Arabidopsis. BMC plant biol. 12, 81 (2012).
33. M. Fendrych et al., The Arabidopsis exocyst complex is involved in cytokinesis and cell plate maturation. Plant Cell 22, 3053 (2010).
34. D. G. Oppenheimer et al., Essential role of a kinesin-like protein in Arabidopsis trichome morphogenesis.Proc. Natl. Acad. Sci. U.S.A. 94, 6261 (1997).
35. J. Riedl et al., Lifeact: a versatile marker to visualize F-actin. Nat, methods 5, 605 (2008).
36. R. Gutierrez, J. J. Lindeboom, A. R. Paredez, A. M. Emons, D. W. Ehrhardt, Arabidopsis cortical microtubules position cellulose synthase delivery to the plasma membrane and interact with cellulose synthase trafficking compartments. Nat. cell biol. 11, 797 (2009).
37. J. Marc et al., A GFP-MAP4 reporter gene for visualizing cortical microtubule rearrangements in living epidermal cells. Plant Cell 10, 1927 (1998).
38. A. Yoneda et al., Chemical genetic screening identifies a novel inhibitor of parallel alignment of cortical microtubules and cellulose microfibrils. Plant Cell Physiol. 48, 1393 (2007).
39. C. M. Hayot, E. Forouzesh, A. Goel, Z. Avramova, J. A. Turner, Viscoelastic properties of cell walls of single living plant cells determined by dynamic nanoindentation. J. exp. bot. 63, 2525 (2012).
40. E. Forouzesh, A. Goel, S. A. Mackenzie, J. A. Turner, In vivo extraction of Arabidopsis cell turgor pressure using nanoindentation in conjunction with finite element modeling. Plant J. 73, 509 (2013).
41. D. B. Szymanski, D. J. Cosgrove, Dynamic coordination of cytoskeletal and cell wall systems during plant cell morphogenesis. Curr. biol.: CB 19, R800 (2009).
42. G. A. Holzapfel, R. W. Ogden, Constitutive modelling of arteries. Proceedings of the Royal Society A: Mathematical, Physical and Engineering Science 466, 1551 (2010).

1. A method for controlling the mechanical properties of the cell wall and polarized growth in a cell, comprising selectively manipulating the cell cytoskeleton.

2. The method of claim 1, the cell is that of a leaf trichome.

3. The method of claim 1, the specific gene type controls properties of tip refinement in the cell.

4. The method of claim 1, further including an engineered protein that modulates the size and position of a microtubule depleted zone, which influences the local properties of the cell wall.

5. The method of claim 1, the specific gene type comprises a genetically engineered protein complex termed the ACTIN-RELATED PROTEIN (ARP) 2/3 that creates actin filaments, the proteins SPIKE 1, PIROGI/SRA1, GNARLED/NAP1, BRICK1/HSPC300, and SCAR/WAVE which regulate ARP2/3, ACTIN which polymerizes to form an actin filament network, and microtubule depleted zone in the cell.
